SEQUENCE LISTING (9 sequences)

SEQ ID NO: 1 - DNA, 576 bp, Homo sapiens
atgctgagttccatcaagtgcgtgttggtgggcgactctgctgtggggaaaacctctctgttggtgcgcttcacctccgagaccttcccggaggcctacaagcccacagtgtacgagaacacaggggtggacgtcttcatggatggcatccagatcagcctgggcctctgggacacagccggcaatgacgccttcagaagcatccggcccctgtcctaccagcaggcagacgtggtgctgatgtgctactctgtggccaaccataactcattcctgaacttgaagaacaagtggattggtgaaattaggagcaacttgccctgtacccctgtgctggtggtggccacccagactgaccagcgggagatggggccccacagggcctcctgcgtcaatgccatggaagggaagaaactggcccaggatgtcagagccaagggctacctggagtgctcagcccttagcaatcggggagtacagcaggtgtttgagtgcgccgtccgaactgccgtcaaccaggccaggagacgaaacagaaggaggctcttctccatcaatgagtgcaagatcttctaa

SEQ ID NO: 2 - protein, 191 aa, Homo sapiens
MLSSIKCVLVGDSAVGKTSLLVRFTSETFPEAYKPTVYENTGVDVFMDGIQISLGLWDTAGNDAFRSIRPLSYQQADVVLMCYSVANHNSFLNLKNKWIGEIRSNLPCTPVLVVATQTDQREMGPHRASCVNAMEGKKLAQDVRAKGYLECSALSNRGVQQVFECAVRTAVNQARRRNRRRLFSINECKIF

SEQ ID NO: 3 - DNA, 24 bp, Artificial Sequence
  Description of Artificial Sequence Synthetic primer
tttggaaacttctccttcacacac

SEQ ID NO: 4 - DNA, 17 bp, Artificial Sequence
  Description of Artificial Sequence Synthetic primer
gcccatccaagcaccgt

SEQ ID NO: 5 - DNA, 18 bp, Artificial Sequence
  Description of Artificial Sequence Synthetic probe
agttgaagactaggcttt

SEQ ID NO: 6 - DNA, 165 bp, Homo sapiens
tggggggtgggggcgtgtgggaggccgagcctgtcctcggatcagttgcgtactctgcccgccccctctgactcatgctgacaatcttcttccttcccctggccacctctctgcccacttggcttcctcagtaccttggtccagctcttcctgcaacggcccagg

SEQ ID NO: 7 - DNA, 687 bp, Homo sapiens
ccactgaatctacatcatttctgttaatatgctaatagcaagggggagggaaaggcatcttactaacccagcgttgaaataatttagtagatgactcaagtctcaaggtttctaagatttactattcatacaattacaattgtcattaagagttttcgctggcgtaagtggcacaacacctttgcaatgagcaaacatagtacttatgcatgagttcagggtacacaagtagttgtcatattatttaaaaaacctcaataaaagaggtctttgttcacaggagaatattaaagtaagctgttcttttgccatatcagagaacatttctgatgaaggtggttttgagaaagtaagtctttgctttttcaccttgacctgaaggggaccgtttgcacagggaatcctggagggggcatgtttagggccaagaaaatgggccttggggctctggctgctcctcgttgcccacggtggttcagttctctctgtggggaggcagcgggagtgacagccaggacagatgaatggggtgaatcttggttctggttttcttaggccatagggtcttatgcaaatcatttaaccagctgaagtctctgtttccttggctgttaaaatagggtttaaagtgtttaccataaagggcagctgtgaggctgtgagatgggagaatcaatctatttattg

SEQ ID NO: 8 - DNA, 27 bp, Artificial Sequence
  Description of Artificial Sequence Synthetic primer
atgggttacaccgacatcgagatgacc

SEQ ID NO: 9 - DNA, 25 bp, Artificial Sequence
  Description of Artificial Sequence Synthetic primer
tctcgtacactgtgggcttgtaggc

(19) United States
(12) Patent Application Publication
(10) Pub. No.: US 2008/0293661 A1
(43) Pub. Date: Nov. 27, 2008

(54) METHOD FOR TREATING CHRONIC LYMPHOID LEUKEMIA

(75) Inventors: Carl Simon SHELLEY, Charlestown, MA (US); Sylvie GALIEGUE-ZOUITINA, Lille (FR)

Correspondence Address: DAVID S. RESNICK, NIXON PEABODY LLP, 100 SUMMER STREET, BOSTON, MA, 02110-2131, US

(73) Assignee: THE GENERAL HOSPITAL CORPORATION, Boston, MA (US)

(21) Appl. No.: 12/123,078

(22) Filed: May 19, 2008

Related U.S. Application Data
(60) Provisional application No. 60/931,360, filed on May 23, 2007.

(51) Int. Cl. A61K 31/711; C12Q 1/02; C12Q 1/68; C12Q 1/66; A61P 35/02

(52) U.S. Cl. 514/44; 435/29; 435/6; 435/8

(57) ABSTRACT

Embodiments of the invention relates to the treatment of chronic lymphoid leukemia. The invention provides treatment, diagnostic, and drug discovery strategies for chronic lymphoid leukemia. Reconstitution of RhoH expression in vitro inhibits neoplastic proliferation and the process of trans-endothelial migration that underlies much of the pathology of CCL. RhoH reconstitution also limits malignant progression in vivo. Therefore, RhoH reconstitution represents a new therapeutic strategy in the fight against chronic lymphoid leukemia.

### CROSS REFERENCE TO RELATED APPLICATIONS

This application claims benefit under 35 U.S.C. § 119(e) of the U.S. Provisional application No. 60/931,360 filed May 23, 2007, the contents of which are incorporated herein by reference in its entirety.

### FIELD OF INVENTION

Embodiments of the invention relates to the treatment of chronic lymphoid leukemia. The invention provides treatment, diagnostic, and drug discovery strategies for chronic lymphoid leukemia.

### BACKGROUND OF THE INVENTION

There are thirteen malignancies that are recognized as chronic lymphoid leukemia. Of these thirteen, the most prevalent types are chronic lymphocytic leukemia (CLL), hairy cell leukemia (HCL), adult T-cell leukemia (ATL) and Sézary syndrome (SzSy). Together, these four types account for over 95% of cases. These different types of chronic lymphoid leukemia are known to coexistance in the same patient, and have to ability to form hybrids and transform from one type to another, indicating the related nature of these different types of chronic lymphoid leukemia.

In the United States, about 30% of all leukemia diagnosed in the United States are of the chronic forms originating from the lymphocyte population of white blood cells. After 30 years of age the rates of occurrence of these chronic lymphoid leukemia rise faster than that of any other types of leukemia. Consequently, chronic lymphoid leukemia of white blood lymphocytes are the most common forms of leukemia diagnosed in people aged 65 and older, affecting 20 out of every 100,000 individuals. As the mean age of the US population continues to rise, chronic lymphoid leukemia will become an increasing challenge to public health. The causes of these chronic lymphoid leukemia are unknown and available treatments are largely ineffective.

ATL is endemic in several regions of the world, particularly Japan, the Caribbean basin and parts of central Africa. ATL is often an extremely aggressive disease, with no characteristic histologic appearance except for a diffuse pattern and a mature T-cell phenotype. Circulating lymphocytes with an irregular nuclear contour are frequently seen. The survival times ranging from 2 weeks to just over a year.

HCL and Sézary syndrome are rarer diseases comprising approximately 3% of lymphoid leukemia. Sézary syndrome is a type of cutaneous lymphoma characterized by Albert Sézary. “Sézary's cells” are T-lymphocytes that have pathological quantities of mucopolysaccharides. Sézary's disease is sometimes considered a late stage of mycosis fungoides. Sézary syndrome is an aggressive disease with an overall survival rate of between 10 and 20% at 5 years.

HCL, is a unique chronic lympho-proliferative disorder characterized by abnormally shaped lymphocytic white blood cells with hair-like projections. These neoplastic lymphocytes move out of circulation and infiltrate organs such as the bone marrow, liver, and spleen. This extravasation leads to peripheral deficiency in circulation blood cells, hepatomegaly, and splenomegaly and compromise of the bone marrow functions. Central to HCL extravasation is the ability of the malignant lymphocytes to first adhere, and then migrate through the vascular endothelium. Such adhesion and migration is facilitated by malignant lymphocytes exhibiting inappropriate expression of the pro-adhesion molecule CD11c. HCL can strike both males and females, usually between the ages of 40 to 70. Prolonged remission of HCL can be achieved using available current treatments, pentostatin and chlorodeoxyadenosine. However, a significant percentage of HCL patients remain resistant to treatment.

Accordingly, in view of the poor treatment options and prognosis of chronic lymphoid leukemia, alternative therapeutic avenues are urgently needed for the treatment and management of chronic lymphoid leukemia.

### SUMMARY OF THE INVENTION

Embodiments of the present invention are based on the discovery that chronic lymphoid leukemia lymphocytes exhibit significantly lower expression of RhoH and that increasing RhoH expression in these lymphocytes reduced cell adhesion, migration and cell proliferation of these lymphocytes in vivo. Hence, chronic lymphoid leukemia is characterized by endogenous RhoH gene repression, and returning RhoH gene expression to normal levels can be useful in limiting neoplastic phenotypes.

Accordingly, in one embodiment, the invention provides a method of treating chronic lymphoid leukemia in a mammal comprising of determining the level of endogenous RhoH gene expression in the lymphocytes of the mammal and reconstituting RhoH gene expression in the chronic lymphoid leukemia lymphocytes if the endogenous RhoH gene expression is below a predetermined level.

In one embodiment, the mammal is a human.

The predetermined level of RhoH is the average of the RhoH level in normal lymphocytes. In chronic lymphoid leukemia lymphocytes, the level of endogeneous RhoH is reduced by 70-99% of the predetermined level of RhoH. The reduction is at least 70%, 80%, 90%, 95% and 99%. In one embodiment, the level of endogeneous RhoH is reduced by at least 70% of the predetermined level. When the endogenous RhoH expression in the clymphocytes is reduced by 70-99% of the predetermined level, reconstituting RhoH gene expression in lymphocytes is performed.

In one embodiment, reconstituting RhoH gene expression in the lymphocytes comprises introducing an exogenous RhoH gene into the lymphocytes. The lymphocytes is a population of neoplastic chronic lymphoid leukemia lymphocytes.

In one embodiment, the exogenous RhoH gene is a RhoH cDNA. In one embodiment, the RhoH cDNA is derived from human. A human Rho H cDNA is the preferred exogenous RhoH cDNA used for the invention, especially when the afflicted mammal is human.

In one embodiment, the exogenous Rho H gene is carried in an expression vector, preferably carried in a mammalian expression vector and the expression is driven by a strong constitutive promoter such as the cytomegalovirus (CMV) promoter or the non-viral EF1α promoter.

In another embodiment, reconstituting the RhoH expression in the lymphocytes comprise administering an exogenous RhoH gene, i.e. a RhoH cDNA to the mammal. Naked RhoH cDNA is administered to the mammal and passive uptake of the naked RhoH cDNA occurs in lymphocytes.

In another embodiment, reconstituting the RhoH gene expression in the lymphocytes comprise administering a vector comprising an exogenous RhoH gene. The vector is an expression vector and functions to express the RhoH protein from the exogenous RhoH gene it comprises. In one embodiment, the expression vector is a viral vector. In an alternate embodiment, the viral vector is a lentiviral vector.

In yet another embodiment, reconstituting the RhoH gene expression in the lymphocytes comprise targeted introduction of an exogenous RhoH gene into the lymphocytes. The neoplastic lymphocytes are harvested and isolated from afflicted patients, and a mammalian expression vector carrying the human RhoH cDNA is introduced into the neoplastic lymphocytes. These transfected lymphocytes carrying the RhoH cDNA are re-introduced into the afflicted patients. In one embodiment, the transfected lymphocytes carrying the RhoH cDNA are re-introduced in conjunction with a bone marrow transplant of the afflicted patient, after radiation and chemotherapy are administered to rid the afflicted mammal of the neoplastic cells.

In one embodiment, the level of endogenous RhoH gene expression is determined by qRT-PCR. A sample of peripheral blood is collect from the afflicted mammal, the lymphocytes from the blood sample are isolated and the endogenous RhoH gene expression is determined by qRT-PCR.

In one embodiment, the chronic lymphoid leukemia is selected from the group consisting of: (a) adult-T-cell leukemia (ATL); (b) chronic lymphocytic leukemia (CLL); (c) hairy cell leukemia (HCL).

In one embodiment, the invention provides a method of diagnosing chronic lymphoid leukemia in a mammal comprising of determining the level of RhoH gene expression in lymphocytes from the mammal, wherein a reduced expression of RhoH compared to a predetermined level indicates presence of chronic lymphoid leukemia. The level of endogenous RhoH gene expression in a mammal suspected of having chronic lymphoid leukemia is determined by qRT-PCR. A mammal is identified as suffering from chronic lymphoid leukemia when the level of endogenous RhoH is reduced by at least 70% of the predetermined level. The predetermined level is the average RhoH expression in the lymphocytes of a normal mammal.

In one embodiment, the invention provides a method of screening for compounds that alleviate RhoH repression in chronic lymphoid leukemia comprising of exposing ATL or HCL derived cell lines to the compounds, determining the level of endogenous RhoH expression in the treated cell lines, and comparing the level with a predetermined level of endogenous RhoH expression, wherein a compound that results in an increase in the endogenous RhoH gene expression in the cell lines is considered a candidate compound for the treatment of chronic lymphoid leukemia. These ATL or HCL derived cell lines carry a CD11c promoter-luciferase reporter vector or a RhoH promoter-luciferase reporter vector. After the compound have interacted with these have ATL or HCL derived cell lines for a period of time, the level of luciferase activity and/or the RhoH expression is determined. In one embodiment, the level of RhoH expression is determined by qRT-PCR.

For cell lines stably carrying the CD11c promoter-luciferase reporter vector, a concurrent increase in endogenous RhoH gene expression and a decrease in the luciferase activity in the compound treated cells indicates that the compound is effective in alleviating the constitutive repression of RhoH expression.

For cell lines stably carrying RhoH promoter-luciferase reporter vector, a concurrent increase in endogenous RhoH gene expression and a increase in the luciferase activity in the compound treated cells indicates that the compound is effective in alleviating the constitutive repression of RhoH expression.

### BRIEF DESCRIPTION OF FIGURES

FIG. 1 shows that RhoH is specifically under-expressed in HCL cell lines. Histograms represent the average relative levels of RhoH expression derived from PCR analysis of two separate RNA preparations. Error bars represent the difference between these two analyses.

FIG. 2 shows that RhoH is under-expressed in HCL patients. Histograms represent the average relative levels of RhoH expression derived from PCR analysis of two separate RNA preparations. Error bars represent the difference between these two analyses.

FIG. 3 shows the repression of RhoH promoter activity in HCL cell line. Each histogram represents the mean ±S.E.M. of three independent experiments.

FIG. 4 shows the effects of RhoH reconstitution in HCL represses CD11c promoter activity. Each histogram represents the mean ±the standard deviation of three independent experiments.

FIG. 5 shows that RhoH reconstitution reduces surface expression of CD11c. Flow cytometric analysis of JOK-1 stably expressing pMEP4 (Empty) and on JOK-1 cells stably expressing pMEP-RhoH (RhoH) are shown. The mean reduction in CD11c expression determined from these experiments was 49.5%±a standard deviation of 9%.

FIG. 6A shows that reconstitution of RhoH Expression in HCL Inhibits homotypic adhesion. Light microscope images of JOK-1 cells that contain in their genome either pMEP4 (Empty) or pMEP4-RhoH (RhoH). Cells were photographed three days after a culture of single cells was initiated in the absence of supplemental CdCl₂.

FIG. 6B shows the percentage of JOK-1 cells either pMEP4 (Empty) or pMEP4-RhoH (RhoH) not present in an aggregate of two or more. Each histogram represents the average of ten microscope fields of 1 mm² acquired from each of two independent experiments. Error bars represent the difference between these two analyses.

FIG. 6C shows the percentage of JOK-1 cells either pMEP4 (Empty) or pMEP4-RhoH (RhoH) present in aggregates classified as “small” (S: 0.1-0.25×10⁴ μm²), “medium” (M: 0.5-1.0×10⁴ μm²) or “large” (L: 1.5-20.0×10⁴ μm²). These values were calculated each day for three days after single-cell cultures were initiated. Each histogram represents the average of six microscope fields of 1 mm² acquired from each of two independent experiments. Error bars represent the difference between these two experiments. Asterisks denote values of 0%.

FIG. 7 shows that RhoH reconstitution in HCL Cells inhibits their adhesion to endothelial cells. Each histogram represents the mean ±the standard deviation of three independent experiments.

FIG. 8 shows that RhoH reconstitution in HCL Cells inhibits their migration through endothelial Cells. Each histogram represents the mean ±the standard deviation of three independent experiments.

FIG. 9 shows that reconstitution of RhoH expression reduces HCL proliferation. Error bars represent the difference between these two experiments.

FIG. 10A shows that RhoH reconstitution inhibits malignant progression of JOK-1 (pMEP) cells and JOK-1 (pMEP-RhoH) in vivo in mice. The mice were classified depending upon whether they had no visible tumor (NG), a “small” tumor of less than 1 cm in maximum diameter (S), a “medium” sized tumor of 1-1.4 cm (M), a “large” tumor of 1.5-2.4 cm (L) or a “very large” tumor of 2.5-3.5 cm (VL).

FIG. 10B shows the histogram of the weight of the spleens isolated from mice injected with RPMI1640 alone (Control) or mice injected with JOK-1 cells expressing either pMEP (Empty) or pMEP-RhoH (RhoH). Histograms depict the mean weight ±S.E.M. of four, nine and twelve mice, respectively.

### DETAILED DESCRIPTION OF THE INVENTION

Unless otherwise stated, the present invention was performed using standard procedures that are well known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.); Current Protocols in Immunology (CPI) (John E. Coligan, et. al., ed. John Wiley and Sons, Inc.); Current Protocols in Cell Biology (CPCB) (Juan S. Bonifacino et. al. ed., John Wiley and Sons, Inc.); Culture of Animal Cells: A Manual of Basic Technique by R. Ian Freshney, Publisher: Wiley-Liss; 5th edition (2005); and Animal Cell Culture Methods (Methods in Cell Biology, Vol 57, Jennie P. Mather and David Barnes editors, Academic Press, 1st edition, 1998), which are all incorporated by reference herein in their entireties.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Definition of terms.

The term “endogenous RhoH” use herein means the RhoH expressed from the original copy of the gene found in the genome of the lymphocytes.

The term “exogenous RhoH” use herein refers to the RhoH expressed from the RhoH cDNA introduced into the lymphocytes. The RhoH cDNA may be carried in a vector or it may be integrated into the genome of the lymphocytes.

The term “constitutive” use herein refers to “all the time” or constantly. For example, a gene product that is made all the time is constitutively expressed. Similarly constantly low expression of a gene means that the gene is constitutively repressed.

The term “neoplastic” use herein refers to new, abnormal, unregulated, disorganized growth or cell division.

The term “vector”, as used herein, refers to a nucleic acid construct designed for transfer between different host cells. An “expression vector” refers to a vector that has the ability to incorporate and express exogenous DNA fragments in a foreign cell. A cloning or expression vector may comprise additional elements, for example, the expression vector may have two replication systems, thus allowing it to be maintained in two organisms, for example in human cells for expression and in a prokaryotic host for cloning and amplification. The term vector may also be used to describe a recombinant virus, e.g., a virus modified to contain the coding sequence for a therapeutic compound or factor. As used herein, a vector may be of viral or non-viral origin.

As used herein, the term “viral vector” is used according to its art-recognized meaning. It refers to a nucleic acid vector construct that includes at least one element of viral origin and may be packaged into a viral vector particle. The vector may be utilized for the purpose of transferring DNA, RNA or other nucleic acids into cells either in vitro or in vivo. Numerous forms of viral vectors are known in the art.

The term “gene” means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

The term “reconstitution” use herein means to bringing the in vivo expression level of RhoH to at least the normal level of a healthy normal lymphocyte. The method of reconstitution is achieved by introducing of a constitutive expression vector carrying a copy of the RhoH cDNA into the cell.

Real-time quantitative reverse transcriptase polymerase chain reaction (qRT-PCR) refers to the reliable detection and measurement of products generated during each cycle of the PCR process which are directly proportionate to the amount of messenger RNA prior to the start of the PCR process.

The term “afflicted” use herein refers to chronic lymphoid leukemia. Afflicted patients refer to patients suffering from chronic lymphoid leukemia.

Unless otherwise indicated, all technical and scientific terms used herein have the same meaning as they would to one skilled in the art of the present invention. Further, unless otherwise required by context, singular terms shall include pluralities and plural terms shall include the singular.

The process of cloning the RhoH cDNA sequence, construction of the RhoH expression vectors and RhoH viral vectors may be performed by conventional recombinant molecular biology and protein biochemistry techniques such as described in Maniatis et. al. (Molecular Cloning—A Laboratory Manual; Cold Spring Harbor, 1982) and DNA Cloning Vols I, II, and III (D. Glover ed., IRL Press Ltd.), Sambrook et. al., (1989, Molecular Cloning, A Laboratory Manual; Cold Spring Harbor Laboratory Press, NY, USA), Current Protocols in Molecular Biology (CPMB) (Fred M. Ausubel, et al. ed., John Wiley and Sons, Inc.) and Current Protocols in Protein Science (CPPS) (John E. Coligan, et. al., ed., John Wiley and Sons, Inc.).

The present invention is based on the discovery that lymphocyte cell lines (Mo, HC-1, EH, ESKOL, JOK-1, JC-1, KK1, SO4, and ST1) derived from patients suffering from chronic lymphoid leukemia exhibit reduced levels of endogenous RhoH gene expression. The cell lines Mo, HC-1, EH, ESKOL, JOK-1, and JC-1 are derived from HCL patients where as the cell lines KK1, SO4, and ST1 are derived from ATL patients. Moreover, the lymphocytes collected from such patients also exhibit constitutively repression of endogenous RhoH gene expression. In addition, these cell lines, and the ATL and HCL lymphocytes harvested from ATL or HCL patients exhibited abnormally enhanced expression of the pro-adhesion protein CD11c. The CD11c protein is a cell surface protein that is responsible for cell-cell adhesion and aggregation, and is normally expressed in cells of the myeloid lineage and in a limited group of activated lymphocytes. The constitutive expression of CD11c in said cell lines and ATL and HCL lymphocytes may contribute to leukemia's shared characteristics of lymphocyte migration and infiltration of organs in afflicted individuals.

Hairy-cell leukemia (HCL) is a chronic lymphoproliferative disease the cause of which is unknown¹⁻³. HCL represents approximately two percent of adult leukemia and is characterized by pancytopenia, hepatomegaly, splenomegaly, leukocytosis and neoplastic mononuclear cells in the peripheral blood, bone marrow, liver and spleen¹⁻³. Purine analogues are the current treatments of choice^{2, 4, 5}. However, a significant proportion of HCL patients remain refractive to these compounds^{4, 5}. In addition, disturbing new evidence has emerged that demonstrates purine analogous only delay disease progression rather than provide a cure⁶. HCL is a disease of late middle-age^{2, 3}. Consequently, in the aging populations of the first and second world, HCL is set to become an increasing health care burden. These considerations highlight the need to develop alternative approaches to the treatment of HCL.

ATL is endemic in several regions of the world, particularly Japan, the Caribbean basin and parts of central Africa. ATL is often an extremely aggressive disease, with no characteristic histologic appearance except for a diffuse pattern and a mature T-cell phenotype. Circulating lymphocytes with an irregular nuclear contour are frequently seen. The survival times ranging from 2 weeks to just over a year.

Patients diagnosed with ATL, HCL or Sézary syndrome present with similar epidemiology, pathogenesis and neoplastic immunophenotype. Patients are predominantly middle-aged, and ATL, HCL and Sézary syndrome can coexist in the same patient. Tumor cells are found in the blood and tumor infiltrates are found in the bone marrow, spleen, liver, lymph nodes and skin. Patients are usually infected with HTLV-I, however, viral infection is not causative. Tumor cells are usually CD4+ T-lymphocytes and patients present with an elevated ratio of CD4+/CD8+ T-lymphocytes.

At the molecular level, ATL, HCL and Sézary syndrome all share some common molecular defects. Tumor cells exhibit abnormal expression of the genes encoding tartrate-resistant acid phosphatase and the proto-oncogene JunD of the Ras family of proto-oncogenes. This Ras family controls the rate at which cells divide and die. The inappropriate expression of Ras causes defective intracellular signaling and consequent aberrant activation of JunD, RhoA, Rac1, Cdc42, p38 MAPK and Akt genes. The inappropriate expression of the pro-adhesion molecular CD11c in HCL is dependent on the activation of the proto-oncogenes Ras and JunD (Nicolaous F. et. al. Blood, 2003, 101:4033-41). Furthermore, inhibition of Ras signaling specifically blocks the proliferation of neoplastic cells, adhesion and trans-endothelial migration of cells. In addition, mutations have been found within and next to Ras genes and the genes encoding the Ras signaling molecules RhoH and NF-kB2 are rearranged.

Normally CD11c gene expression is restricted to cells of the myeloid lineage and to a limited group of activated lymphocytes¹¹. However, in HCL CD11c is constitutively expressed on the surface of the neoplastic lymphocytes and represents a diagnostic marker for the disease 9. Previous studies have established that constitutive expression of the CD11c gene in hairy-cells is directed by the promoter region extending from 128 bp upstream to 36 bp downstream of the 5′ major transcription initiation site¹³.

Within the CD11c promoter numerous individual cis-acting control elements have been identified^{13, 53, 58}. Mutation of these elements has established that the element most critical to the transcriptional activity of the CD11c promoter in hairy-cells interacts with the AP-1 family of transcription factors¹³. Further analyses demonstrated that hairy-cells exhibit abnormal constitutive expression of AP-1 mediated by activation of the Ras family of proto-oncogenes 13 and the inventors discovered that RhoH plays a key role in this process.

RhoH is expressed exclusively in white blood cells and acts as a natural dominant-negative regulator of Ras signaling pathways^{38, 39}. In normal resting leukocytes the RhoH gene is expressed at high levels limiting survival, proliferation and adhesion. When normal leukocytes are activated, transcription of the RhoH gene is repressed causing survival, proliferation and adhesion to increase³⁹.

Cell proliferation is driven by Ras activation⁵⁹. Since RhoH is a negative regulator of the Ras family this provides a molecular explanation for RhoH reconstitution being able to inhibit HCL proliferation.

The malignant cells of HCL can be derived both from B and T lymphocytes as demonstrated by their expression of B or T cell specific antigens^{7, 8}. However, biochemically, HCL is diagnosed by expression of tartrate resistant acid phosphatase and the β2-integrin CD11c that are usually expressed predominantly by myeloid cells^{9, 12}. Critical to the expression of the CD11c gene is its interaction with the transcription factor AP-1¹³. Normally, AP-1 expression is transient and mediates induction of CD11c in response to specific myeloid differentiation signals¹⁴. However, in the neoplastic lymphocytes of HCL AP-1 is expressed constitutively thus driving abnormal concomitant expression of CD11c¹³. This abnormality in quantitative AP-1 expression is also linked to an abnormality that is qualitative. Normally, AP-1 induced in non-hairy-cells by signals of differentiation or proliferation consists of a complex mixture of homo and heterodimers composed of different members of the Jun and Fos families of proto-oncogenes¹³. However, in HCL the AP-1 complex that is expressed constitutively appears to consist only of homodimers of JunD 13. Members of the Jun family such as JunD are controlled by mechanisms that act both at the transcriptional and post transcriptional level¹⁵⁻³⁰. One of the most important post-transcriptional mechanisms is protein phosphorylation regulated by the Ras family of proto-oncogenes³¹⁻³³. The Ras family can transform fibroblasts in cooperation with JunD and H-Ras has been shown to be chronically expressed in the hairy-cell line ESKOL^{34, 35}. Previous studies have shown the role of Ras in HCL and CD11c expression first by inhibiting its expression with the dominant negative mutant RasN17¹³. The use of this mutant in transient transfection assays inhibited CD11c promoter activity in hairy-cells. Consequently, Ras activation appears to contribute to hairy-cell expression of CD11c. Next, we expressed in non-hairy-cells the Ras mutant. Exogenous expression of this dominant positive mutant RasV12¹³ induced the activity of the CD11c promoter. Consequently, Ras activation appears both necessary and sufficient to induce the CD11c promoter.

Here, the inventors discovered that this mechanism involves a comparatively new member of the Ras superfamily named RhoH^{36, 37}. This molecule is unique within the Ras family in being expressed exclusively by hematopoietic cells and in lacking guanosine triphosphatase (GTPase) activity^{38, 39}. Due to its inability to hydrolyze guanosine triphsphate (GTP), RhoH remains GTP bound and so acts as a non-competitive inhibitor of members of the Ras family that cycle between forms bound by GTP and guanosine diphosphate (GDP)³⁸. The inhibitory nature of RhoH is manifest at the cellular level by it mediating increased apoptosis and reduced proliferation and adhesion of murine hematopoietic progenitor cells³⁹. In contrast, knockdown of RhoH in progenitor cells by RNA interference has been shown to increase survival, proliferation and adhesion³⁹. The inventors demonstrated that RhoH exhibits chronic under-expression in cell lines derived from patients with HCL but not in cell lines derived from other forms of leukemia. Subsequently, the inventors confirmed this finding by analysis of lymphocytes isolated directly from patients with HCL and from normal control individuals. Since RhoH repression has been found to increase cell proliferation and adhesion, we reasoned that low expression of RhoH in HCL might lead to similar consequences³⁹. In order to test this hypothesis, the inventors generated pools of HCL cells that contain genomic integration of either an empty expression plasmid or this same plasmid constitutively expressing RhoH. Comparison of these two pools of cells demonstrated that reconstitution of RhoH expression reduces proliferation and both homotypic and heterotypic adhesion. RhoH reconstitution also reduced the process of trans-endothelial migration that is central to HCL pathogenesis. Finally, in an in vivo xenograft mouse model of HCL the inventors found that RhoH reconstitution inhibited malignant progression and protected against mortality. These findings thus provide proof-of-principle that RhoH reconstitution represents a promising new therapeutic strategy to combat HCL.

Since RhoH normally functions to limit cell proliferation and survival, chronic under-expression of RhoH in HCL might contribute to disease progression by increasing the rate of cell division and the lifespan of the neoplastic lymphocytes. HCL is also characterized by infiltration of circulating neoplastic cells into a variety of tissues¹⁻³. Infiltration is dependent upon the ability of HCL cells to bind and migrate through the vascular endothelium. Therefore, in addition to facilitating cell proliferation, RhoH under-expression might also contribute to the pathogenesis of HCL by stimulating lymphocyte adhesion and trans-endothelial migration.

In the example, in order to alleviate the RhoH gene repression and restore normal RhoH expression to the HCL cell lines, a constitutive expression vector carrying a copy of the human RhoH cDNA (Genebank Accession No. AF498975) (SEQ. ID. No. 1) was introduced into the cell lines by electroporation.

The human RhoH cDNA nucleic acid sequence is: 5′-

(SEQ. ID. No. 1)

ATGCTGAGTTCCATCAAGTGCGTGTTGGTGGGCGACTCTGCTGTGGGGAA
AACCTCTCTGTTGGTGCGCTTCACCTCCGAGACCTTCCCGGAGGCCTACA
AGCCCACAGTGTACGAGAACACAGGGGTGGACGTCTTCATGGATGGCATC
CAGATCAGCCTGGGCCTCTGGGACACAGCCGGCAATGACGCCTTCAGAAG
CATCCGGCCCCTGTCCTACCAGCAGGCAGACGTGGTGCTGATGTGCTACT
CTGTGGCCAACCATAACTCATTCCTGAACTTGAAGAACAAGTGGATTGGT
GAAATTAGGAGCAACTTGCCCTGTACCCCTGTGCTGGTGGTGGCCACCCA
GACTGACCAGCGGGAGATGGGGCCCCACAGGGCCTCCTGCGTCAATGCCA
TGGAAGGGAAGAAACTGGCCCAGGATGTCAGAGCCAAGGGCTACCTGGAG
TGCTCAGCCCTTAGCAATCGGGGAGTACAGCAGGTGTTTGAGTGCGCCGT
CCGAACTGCCGTCAACCAGGCCAGGAGACGAAACAGAAGGAGGCTCTTCT
CCATCAATGAGTGCAAGATCTTCTAA-3′.

The human RhoH has the amino acid sequence is:

(SEQ. ID. No. 2)

MLSSIKCVLVGDSAVGKTSLLVRFTSETFPEAYKPTVYENTGVDVFMDGI
QISLGLWDTAGNDAFRSIRPLSYQQADVVLMCYSVANHNSFLNLKNKWIG
EIRSNLPCTPVLVVATQTDQREMGPHRASCVNAMEGKKLAQDVRAKGYLE
CSALSNRGVQQVFECAVRTAVNQARRRNRRRLFSINECKIF
(Genebank Accesion No. AAM21122)

The upstream strong CMV promoter drives the constant expression of human RhoH in the transfected cell lines. The reconstitution of RhoH expression in the cell lines in vitro reduces CD11c expression, inhibited cell adhesion and migration, and the cell proliferation of xenographs of transfected cell lines in vivo.

Accordingly, embodied in the invention is a method of treating chronic lymphoid leukemia in a mammal comprising of determining the level of endogenous RhoH gene expression in the lymphocytes of a mammal and reconstituting the RhoH gene expression in the lymphocytes if the endogenous RhoH gene expression is below a predetermined level, for example, the level found in normal lymphocytes. The mammal can be a human, cat, or dog.

In one embodiment, peripheral blood is collected from the afflicted patients and healthy volunteers by one skilled in the art and lymphocytes are separated from the other blood cells by standard separation techniques that are known to those skilled in the art, preferably by density gradient centrifugation. Other methods include automated cell sorting machines used in routine complete blood count (CBC) test. Total RNA is harvested by methods that are known in the art, such as by Trizol reagent. The level of endogenous Rho H gene expression is determined by qRT-PCR using pair of specially designed primers that will amplify all the variant forms of the RhoH transcripts. The ABL gene transcript serves as an experimental control and for normalizing the data. The sense RhoH primer used is 5′-TTTGGAAACTTCTCCTTCACACAC-3′ (SEQ. ID. No. 3) and the anti-sense RhoH primer 5′-GCCCATCCAAGCACCGT-3′ (SEQ. ID. No. 4). The size of the PCR product resulting from the use of these primers is 171 base-pair (bp) and, therefore, compatible with good amplification efficiency. The RhoH probe is 5′-AGTTGAAGACTAGGCTTT-3′ (SEQ. ID. No. 5) and is labeled at its 5′-end with 6-carboxy-fluorescein phosphoramidite (FAM) and at its 3′-end with non-fluorescent quencher (NFQ). The ABL probe that is used as control for cDNA quantity and quality is also labeled with FAM at its 5′-end but with 6-carboxy-tetramethyl-rhodamine (TAMRA) as quencher at its 3′-end. The relative level of endogenous RhoH expression is then calculated by dividing the quantitative level of endogenous RhoH expression by the quantitative level of endogenous ABL expression obtained for the same total RNA sample.

In one embodiment, the predetermined level of RhoH is the average of the RhoH level in normal lymphocytes. In chronic lymphoid leukemia lymphocytes, the level of endogeneous RhoH is reduced by 70-99% of the predetermined level of RhoH. The reduction can be at least 70%, 80%, 90%, 95% and 99%, including all the percentages between 70% and 99%. In one embodiment, the RhoH gene expression is reconstituted by introducing an exogenous RhoH cDNA into a population of neoplastic chronic lymphoid leukemia lymphocytes.

In one embodiment, reconstituting the RhoH gene expression in the lymphocytes comprises introducing an exogenous RhoH gene into lymphocytes. In one embodiment, the exogenous RhoH gene is a RhoH cDNA. In one embodiment, the RhoH cDNA is derived from human. A human Rho H cDNA is the preferred exogenous RhoH cDNA used for the invention, especially when the afflicted mammal is human. The human RhoH cDNA can be carried in a mammalian expression vector and the expression should be driven by a strong constitutive promoter such as the cytomegalovirus (CMV) promoter or the non-viral EF1α promoter. Examples of suitable mammalian expression vectors include pcDNA3 and pEF-DESTSI from Invitrogen, Inc.

In another embodiment, reconstituting the RhoH gene expression in the lymphocytes comprises administering an exogenous RhoH gene, i.e. a RhoH cDNA to the mammal. Naked RhoH cDNA is administered to the mammal and passive uptake of the naked RhoH cDNA occurs in lymphocytes.

In another embodiment, reconstituting the RhoH gene expression in the lymphocytes comprises administering a vector comprising an exogenous RhoH gene. The vector is an expression vector and it expresses the RhoH protein from the exogenous RhoH gene it comprise. In one embodiment, the expression vector is a viral vector. Viral vectors have the added advantage of facilitating the introduction of exogenous DNA into a cell.

In yet another embodiment, reconstituting the RhoH gene expression in the lymphocytes comprise targeted introduction of an exogenous RhoH gene into lymphocytes.

In one embodiment, the neoplastic lymphocytes are harvested and isolated from patients, and a mammalian expression vector carrying the human RhoH cDNA is introduced into the neoplastic lymphocytes by electroporation or by Biolistic® particle bombardment (Biorad's Helios Gene Gun). Other methods of transfecting cell with exogenous RhoH gene is contemplated and are well known one skilled in the art. The exogenous RhoH gene can also be introduced by a modified virus carrying the RhoH cDNA. The neoplastic lymphocytes can be transfected with an expression vector carrying the human RhoH cDNA by any means known to one skilled in the art. These transfected lymphocytes can then be cultured and expanded for at least 3 doubling cycles and up to 10 doubling cycles in vitro by tissue culture techniques known in the art before they are harvested, filtered, and re-introduced back into circulation in the afflicted patient by intravenous injection in a sterile saline solution. During the in vitro expansion period, the RhoH expression level in these transfected lymphocytes should be monitored by qRT-PCT to determine that the expression vector had been introduced and that the expression vector is expressing RhoH in sufficient quantities. The transfected lymphocyte should have a RhoH level that is a range of 70-95% of a predetermined RhoH level in normal healthy lymphocytes, wherein the predetermined level is the average RhoH found in normal lymphocytes. In addition, a sample of the transfected lymphocytes can be cryo-preserved for the future use. The transfected lymphocytes can be cryo-preserved by any methods that are well known in the art, such as U.S. Pat. No. 7,112,576 and WO/2001/045503 and these are hereby incorporated by reference in their entirety.

In one embodiment, the expression vector is a viral vector. In an alternate embodiment, the viral vector is a lentiviral vector. There are many examples of the use of lentiviral vectors for gene therapy, such as for the treatment of inherited disorders of haematopoietic cells and various types of cancer, and they are hereby incorporated by reference (Klein, C. and Baum, C. (2004). Hematol. J., 5, 103-111; Zufferey, R et. al. (1997). Nat. Biotechnol., 15, 871-875; Morizono, K. et. al. (2005). Nat. Med., 11, 346-352; Di Domenico, C. et. al. (2005). Hum. Gene Ther., 16, 81-90). Other types of viral vectors include but are not limited to adenoviral vectors and adenovirus-associated viral vectors.

In another embodiment, the exogenous RhoH cDNA is delivered to the chronic lymphoid leukemia lymphocytes in vivo by target-based nanoparticles. Cationic lipid-based nanoparticles such as those made of N-(1-(2,3-dioleoyloxy)propyl),N,N,N,-trimethylammonium chloride:cholresterol (DOTAP:cholesterol) complexed with the exogenous RhoH cDNA within (Ito I, et. al. 2004 Cancer Gene Ther. 11:733-9). The modified cholesterol may contain targeting components such as hypervariable region of anti-CD11c antibodies for targeting to said lymphocytes with the overexpression of cell surface protein CD11c Alternatively, the nanoparticles may comprise of hybrid, amino-functionalized organically modified silica (ORMOSIL) (Bharali D J., et. al. 2005, Proc Natl Acad Sci USA. 102:11539-44). The general procedure for the preparation and characterization of ORMOSIL/DNA nanoparticles are describe by Roy I. et. al. 2005, Proc Natl Acad Sci USA. 102:279-284, and Roy, I. et. al., 2003, J. Am. Chem. Soc. 125:7860-7865, and are hereby incorporated in reference. The RhoH cDNA carrying vector in mixed with ORMOSIL and incubated for 30 minutes at room temperature. The resulting ORMOSIL/RhoH vector is then suspended in sterile phosphate buffered saline with a vector DNA concentration of at least 100 μg/ml, preferably in the range between 135-200 μg/ml. The nanoparticle complex is filter-sterilized before introducing into the patient by intravenous injection.

In another embodiment, the lymphocytes transfected with an exogenous RhoH cDNA is re-introduced to the afflicted mammal as part of a bone-marrow transplantation procedure that are well known to one skilled in the art, such as described in U.S. Pat. Nos. 4,486,188, 5,806,529, 6,461,869, and 6,551,589. Radiation and chemotherapy are administered to rid the afflicted mammal of the neoplastic cells. The Rho H cDNA transfected lymphocyes can be mixed with donor bone marrow and administered to the afflicted mammal.

In one embodiment, the types of chronic lymphoid leukemia that are treated by the invention include adult-T-cell leukemia (ATL), chronic lymphocytic leukemia (CLL), and hairy cell leukemia (HCL). These leukemia can be diagnosed in human, but can also be found in pets such as cats and dogs.

In one embodiment, the treatment of chronic lymphoid leukemia is combined with other treatments for adult-T-cell leukemia (ATL), chronic lymphocytic leukemia (CLL), and hairy cell leukemia (HCL).

In another embodiment, the invention provides for a method of diagnosing chronic lymphoid leukemia in a mammal comprising of determining the level of Rho H expression in the lymphocytes, wherein a reduced level expression of Rho H indicates presence of chronic lymphoid leukemia. In one embodiment, the level of endogenous Rho H gene expression is determined by qRT-PCR. In chronic lymphoid leukemia lymphocytes, the level of endogenous RhoH is reduced by at least 70% of the predetermined level of RhoH. The predetermined level of RhoH is the average of the RhoH levels in lymphocytes found in a normal mammal. The reduction can be at least 70%, 80%, 90%, 95% and 99%, including all the percentages between 70% and 99% the predetermined level of RhoH.

Embodied in the invention are reporter cell lines derived from patients with chronic lymphoid leukemia. These cell lines are stably transfected with a luciferase reporting gene. These cell lines make useful tools for the drug discovery and drug efficacy screening process in the search for additional treatment options for chronic lymphoid leukemia. The ATL or HCL derived cell lines such as Mo, HC-1, EH, ESKOL, JOK-1, JC-1, KK1, SO4, and ST1 can be stably transfected with a CD11c promoter-luciferase reporter vector or a RhoH promoter-luciferase reporter vector. For the CD11c promoter-luciferase reporter vector, a 166 bp fragment of the human CD11c gene (Genbank Accession No. NM_{—}000887; XM_{—}937997) corresponding to 128 bp of the 5′UTR and the first 36 bp in the coding region (−128 to +36) is ligated into a promotorless firely luciferase reporter vector pATLuc.

The 166 bp fragment of the human CD11c promoter region is

(SEQ. ID. No. 6)

TGGGGGGTGGGGGCGTGTGGGAGGCCGAGCCTGTCCTCGGATCAGTTGCG
TACTCTGCCCGCCCCCTCTGACTCATGCTGACAATCTTCTTCCTTCCCCT
GGCCACCTCTCTGCCCACTTGCTTCCTCAGTACCTTGGTCCAGCTCTTCC
TGCAACGGCCCAGG

For the RhoH promoter-luciferase reporter vector, a 5′ UTR 650 bp fragment of the human Rhoh gene (SEQ. ID. No. 6) and the first 36 bp coding region of exon 1a is ligated into pATLuc. The respective vectors are introduced into the individual cell lines by electroporation.

The human Rho H gene promoter region, 650 bp upstream of the exon 1a, and includes 36 bp of the exon 1a is 5′-

(SEQ. ID. No. 7)

CCACTGAATCTACATCATTTCTGTTAATATGCTAATAGCAAGGGGGAGGG
AAAGGCATCTTACTAACCCAGCGTTGAAATAATTTAGTAGATGACTCAAG
TCTCAAGGTTTCTAAGATTTACTATTCATACAATTACAATTGTCATTAAG
AGTTTTCGCTGGCGTAAGTGGCACAACACCTTTGCAATGAGCAAACATAG
TACTTATGCATGAGTTCAGGGTACACAAGTAGTTGTCATATTATTTAAAA
AACCTCAATAAAAGAGGTCTTTGTTCACAGGAGAATATTAAAGTAAGCTG
TTCTTTTGCCATATCAGAGAACATTTCTGATGAAGGTGGTTTTGAGAAAG
TAAGTCTTTGCTTTTTCACCTTGACCTGAAGGGGACCGTTTGCACAGGGA
ATCCTGGAGGGGGCATGTTTAGGGCCAAGAAAATGGGCCTTGGGGCTCTG
GCTGCTCCTCGTTGCCCACGGTGGTTCAGTTCTCTCTGTGGGGAGGCAGC
GGGAGTGACAGCCAGGACAGATGAATGGGGTGAATCTTGGTTCTGGTTTT
CTTAGGCCATAGGGTCTTATGCAAATCATTTAACCAGCTGAAGTCTCTGT
TTCCTTGGCTGTTAAAATAGGGTTTAAAGTGTTTACCATAAAGGGCAGCT
GTGAGGCTGTGAGATGGGAGAATCAATCTATTTATTG-3′

Embodied in the invention is a method of screening for compounds that alleviate RhoH repression in chronic lymphoid leukemia comprising of exposing ATL or HCL derived reporter cell lines described herein to the compounds, determining the level of endogenous RhoH expression in the cell lines, and comparing the level with the predetermined level of endogenous RhoH. In one embodiment, the level of RhoH gene expression is determined by qRT-PCR. In addition, the method further comprises determining the level of luciferase activities in compound treated and untreated cells, and comparing the luciferase activities in compound treated and untreated cells.

For cell lines stably carrying the CD11c promoter-luciferase reporter vector, a concurrent increase in endogenous RhoH gene expression and a decrease in the luciferase activity in the compound treated cells indicates that the compound is effective in alleviating the constitutive repression of RhoH expression.

For cell lines stably carrying RhoH promoter-luciferase reporter vector, a concurrent increase in endogenous RhoH gene expression and a increase in the luciferase activity in the compound treated cells indicates that the compound is effective in alleviating the constitutive repression of RhoH expression. Such a compound that results in an increase in the endogenous RhoH gene expression in the cell lines is considered a candidate compound for the treatment of chronic lymphoid leukemia.

Compound libraries of over 130,000 compounds from ChemDiv SPECS & BioSPECS, Chembridge, ChemRX, LOPAC, and NCl DTP can be screened in High-Throughput Screening (HTS) assays that are well known to one skilled in the art. For example, U.S. Pat. Nos. 6,830,897, 6,936,462, and WO/2004/018632, and these are hereby incorporated by reference in their entirety. Other libraries useful to screen include but are not limited to compound library from Prestwick Chemicals, TimTec, and TimTec Library.

In one embodiment, the method described herein encompasses a pharmaceutical composition comprising an expression vector comprising a nucleic acid encoding a RhoH cDNA (exogenous RhoH gene) and a pharmaceutically acceptable carrier.

The isolated nucleic acid sequences of RhoH cDNA, RhoH promoter, and the CD11 promoter sequences (SEQ. ID. No. 1, 6 and 7) are isolated and amplified using a number of standard techniques that are well known to one skilled in the art, such as RT-PCR.

Once ligated into a vector, the nucleic acid can be subcloned into several expression vectors, such as a viral expression vector or a mammalian expression vector by PCR cloning, restriction digestion followed by ligation, or recombination reaction such as those of the lambda phage-based site-specific recombination using the Gateway® LR and BP Clonase™ enzyme mixtures. Subcloning should be unidirectional such that the 5′ transcription start nucleotide of the nuclei acid sequence is downstream of the promoter in the expression vector. Alternatively, when the nucleic acid sequence is cloned into pENTR/D-TOPO®, pENTR/SD/D-TOPO® (directional entry vectors), or any of the Invitrogen's Gateway® Technology pENTR (entry) vectors, the nucleic acid sequence can be transferred into the various Gateway® expression vectors (destination) for protein expression in host cells in one single recombination reaction. Some of the Gateway® destination vectors are designed for the constructions of baculovirus, adenovirus, adeno-associated virus (AAV), retrovirus, and lentiviruses, which upon infecting their respective host cells, facilitating ease of introducing the transgene into the host cells. The Gateway® Technology uses lambda phage-based site-specific recombination instead of restriction endonuclease and ligase to insert a gene of interest into an expression vector. The DNA recombination sequences (attL, attR, attB, and attP) and the LR and BP Clonase™ enzyme mixtures that mediate the lambda recombination reactions are the foundation of Gateway® Technology. Transferring a gene into a destination vector is accomplished in just two steps: Step 1: Clone the nucleic acid sequence of interest into an entry vector such as pENTR/D-TOPO®. Step 2: Mix the entry clone containing the nucleic acid sequence of interest in vitro with the appropriate Gateway® expression vector (destination vector) and Gateway® LR Clonase™ enzyme mix. There are Gateway® expression vectors for protein expression in E. coli, insect cells, mammalian cells, and yeast. Site-specific recombination between the att sites (attR×attL and attB×attP) generates an expression vector and a by-product. The expression vector contains the nucleic acid sequence of interest recombined into the destination vector backbone. Following transformation and selection in E. coli, the expression vector is ready to be used for expression in the appropriate host.

In one embodiment, the RhoH cDNA (exogenous RhoH gene) is expressed from a recombinant circular or a linear DNA vector using any suitable promoter. Suitable promoters for expressing RNA from a vector include, e.g., the U6 or H1 RNA pol III promoter sequences, or the cytomegalovirus promoters. Selection of other suitable promoters is within the skill in the art. The expression vector should have the necessary 5′ upstream and 3′ downstream regulatory elements such as promoter sequences, ribosome recognition and binding TATA box, and 3′ UTR AAUAAA transcription termination sequence for the efficient gene transcription and translation in its respective host cell. The recombinant vectors can also comprise inducible or regulatable promoters for expression of the nucleic acid sequence of interest.

Selection of vectors suitable for expressing the nucleic acid sequence, methods for inserting nucleic acid sequences into vector to express the gene products, and methods of delivering the recombinant vector to the cells of interest are within the skill in the art. See, for example, Zeng et al. (2002), Molecular Cell 9:1327-1333; Tuschl (2002), Nat. Biotechnol, 20:446-448; Brummelkamp et al. (2002), Science 296:550-553; Miyagishi et al. (2002), Nat. Biotechnol. 20:497-500; Paddison et al. (2002), Genes Dev. 16:948-958; Lee ei al. (2002), Nat. Biotechnol. 20:500-505; and Paul et al. (2002), Nat. Biotechnol. 20:505-508, the entire disclosures of which are incorporated herein by reference in their entirety.

Examples of expression vectors for mammalian host cells include but are not limited to the strong CMV promoter-based pcDNA3.1 (Invitrogen) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech), pAd/CMV/V5-DEST, pAd-DEST vector (Invitrogen) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells; adenovirus-associated virus expression vectors such as pAAV-MCS, pAAV-IRES-hrGFP, and pAAV-RC vector (Stratagene) for adeno-associated virus-mediated gene transfer and expression in mammalian cells;

A simplified system for generating recombinant adenoviruses is presented by He T C. et. al. Proc. Natl. Acad. Sci. USA 95:2509-2514, 1998. The gene of interest is first cloned into a shuttle vector, e.g. pAdTrack-CMV. The resultant plasmid is linearized by digesting with restriction endonuclease Pme I, and subsequently cotransformed into E. coli. BJ5183 cells with an adenoviral backbone plasmid, e.g. pAdEasy-1 of Stratagene's AdEasy™ Adenoviral Vector System. Recombinant adenovirus vectors are selected for kanamycin resistance, and recombination confirmed by restriction endonuclease analyses. Finally, the linearized recombinant plasmid is transfected into adenovirus packaging cell lines, for example HEK 293 cells (E1-transformed human embryonic kidney cells) or 911 (E1-transformed human embryonic retinal cells) (Human Gene Therapy 7:215-222, 1996). Recombinant adenovirus are generated within the HEK 293 cells.

In one embodiment, a recombinant lentivirus is used for the delivery and expression of a RhoH cDNA in either dividing and non-dividing mammalian cells. The HIV-1 based lentivirus can effectively transduce a broader host range than the Moloney Leukemia Virus (MoMLV)-base retroviral systems. Preparation of the recombinant lentivirus can be achieved using the pLenti4/V5-DEST™, pLenti6/V5-DEST™ or pLenti vectors together with ViraPower™ Lentiviral Expression systems from Invitrogen.

In one embodiment, a recombinant adeno-associated virus (rAAV) vector is used for the expression of a RhoH cDNA. Because AAV is non-pathogenic and does not illicit an immune response, a multitude of pre-clinical studies have reported excellent safety profiles. rAAVs are capable of transducing a broad range of cell types and transduction is not dependent on active host cell division. High titers, >10⁸ viral particle/ml, are easily obtained in the supernatant and 10¹¹-10¹² viral particle/ml with further concentration. The transgene is integrated into the host genome so expression is long term and stable.

The use of alternative AAV serotypes other than AAV-2 (Davidson et al (2000), PNAS 97(7)3428-32; Passini et al (2003), J. Virol 77(12):7034-40) has demonstrated different cell tropisms and increased transduction capabilities. With respect to brain cancers, the development of novel injection techniques into the brain, specifically convection enhanced delivery (CED; Bobo et al (1994), PNAS 91(6):2076-80; Nguyen et al (2001), Neuroreport 12(9):1961-4), has significantly enhanced the ability to transduce large areas of the brain with an AAV vector.

Large scale preparation of AAV vectors is made by a three-plasmid cotransfection of a packaging cell line: AAV vector carrying the DNA coding sequence, AAV RC vector containing AAV rep and cap genes, and adenovirus helper plasmid pDF6, into 50×150 mm plates of subconfluent 293 cells. Cells are harvested three days after transfection, and viruses are released by three freeze-thaw cycles or by sonication.

AAV vectors are then purified by two different methods depending on the serotype of the vector. AAV2 vector is purified by the single-step gravity-flow column purification method based on its affinity for heparin (Auricchio, A., et. al., 2001, Human Gene therapy 12; 71-6; Summerford, C. and R. Samulski, 1998, J. Virol. 72:1438-45; Summerford, C. and R. Samulski, 1999, Nat. Med. 5: 587-88). AAV2/1 and AAV2/5 vectors are currently purified by three sequential CsCl gradients. Delivery vectors can also included but are not limited to replication-defective adenoviral vectors, cationic liposomes and protein-cationic peptides. For example, one study reports a system to deliver DNA in vitro by covalently attaching the surfactant associated protein B (SP-B) to a 10 kDa poly-lysine. See, Baatz, J., et al., PNAS USA, 91:2547-2551 (1994). See, e.g., Longmuir, et al., 1992 ASBMB/Biophysical Society abstract; Longmuir, et al., 1993 Biophysical Society abstract.

In one embodiment, the pharmaceutical compositions described herein is administered systemically in a pharmaceutical formulation. Systemic routes include but are limited to oral, parenteral, nasal inhalation, intratracheal, intrathecal, intracranial, and intrarectal. The pharmaceutical formulation is preferably a sterile saline or lactated Ringer's solution. For therapeutic applications, the preparations described herein are administered to a mammal, preferably a human, in a pharmaceutically acceptable dosage form, including those that may be administered to a human intervenously as a bolus or by continuous infusion over a period of time, by intramuscular, intraperitoneal, intracerebrospinal, subcutaneous, intra-arterial, intrasynovial, intrathecal, oral, topical, or inhalation routes. The compositions described herein are also suitably administered by intratumoral, peritumoral, intralesional or perilesional routes, to exert local as well as systemic effects. The intraperitoneal route is expected to be particularly useful, for example, in the treatment of ovarian tumors. For these uses, additional conventional pharmaceutical preparations such as tablets, granules, powders, capsules, and sprays may be preferentially required. In such formulations further conventional additives such as binding-agents, wetting agents, propellants, lubricants, and stabilizers may also be required.

In one embodiment, the pharmaceutical compositions described herein are formulated in a cationic liposome formulation. Liposomes, spherical, self-enclosed vesicles composed of amphipathic lipids, have been widely studied and are employed as vectors for in vivo administration of therapeutic agents. In particular, the so-called long circulating liposomes formulations which avoid uptake by the organs of the mononuclear phagocyte system, primarily the liver and spleen, have found commercial applicability. Such long-circulating liposomes include a surface coat of flexible water soluble polymer chains, which act to prevent interaction between the liposome and the plasma components which play a role in liposome uptake. Alternatively, hyaluronan has been used as a surface coating to maintain long circulation. Vector DNA and/or virus can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et. al. Gene Ther. 1999, 6:1438-47). The liposomes can comprise multiple layers assembled in a step-wise fashion. Other techniques in formulating expression vectors and virus as therapeutics are found in “DNA-Pharmaceuticals: Formulation and Delivery in Gene Therapy, DNA Vaccination and Immunotherapy” by Martin Schleef (Editor) December 2005, Wiley Publisher, and “Plasmids for Therapy and Vaccination” by Martin Schleef (Editor) May 2001, are incorporated herein as reference. In one embodiment, the dosage for viral vectors is 10⁶ to 1×10¹⁴ viral vector particles per application per patient.

Lipid materials well known and routinely utilized in the art to produce liposomes. Lipids may include relatively rigid varieties, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, it is preferred to add an additional lipid component, such as cholesterol. Preferred lipids for producing liposomes according to the invention include phosphatidylethanolamine (PE) and phosphatidylcholine (PC) in further combination with cholesterol (CH). According to one embodiment of the invention, a combination of lipids and cholesterol for producing the liposomes of the invention comprise a PE:PC:Chol molar ratio of 3:1:1. Further, incorporation of polyethylene glycol (PEG) containing phospholipids is also contemplated by the present invention.

In addition, in order to prevent the uptake of the liposomes into the cellular endothelial systems and enhance the uptake of the liposomes into the tissue of interest, the outer surface of the liposomes may be modified with a long-circulating agent. The modification of the liposomes with a hydrophilic polymer as the long-circulating agent is known to enable to prolong the half-life of the liposomes in the blood

Liposomes encapsulating the nucleic acid sequences described herein can be obtained by any method known to the skilled artisan. For example, the liposome preparation of the present invention can be produced by reverse phase evaporation (REV) method (see U.S. Pat. No. 4,235,871), infusion procedures, or detergent dilution. A review of these and other methods for producing liposomes may be found in the text Liposomes, Marc Ostro, ed., Marcel Dekker, Inc., New York, 1983, Chapter 1. See also Szoka Jr. et al., (1980, Ann. Rev. Biophys. Bioeng., 9:467).

The compositions can be formulated as a sustained release composition. For example, sustained-release means or delivery devices are well known in the art and they include, but are not limited to, sustained-release matrices such as biodegradable matrices or semi-permeable polymer matrices in the form of shaped articles, e.g., films, or microcapsules that comprise RhoH cDNA expression vectors.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

Sustained-release matrices include polylactides (U.S. Pat. No. 3,773,919, EP 58,481), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release compositions also include liposomally entrapped the RhoH cDNA expression vectors, and nucleic acid constructs. Such liposomes can be prepared by methods known per se: DE 3,218,121; Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy. Other biodegradable polymers and their use are described, for example, in detail in Brem et al. (1991, J. Neurosurg. 74:441-446).

Methods for preparing liposomes and microspheres for administration to a patient are well known to those of skill in the art. U.S. Pat. No. 4,789,734, the contents of which are hereby incorporated by reference, describes methods for encapsulating biological materials in liposomes. A review of known methods is provided by G. Gregoriadis, Chapter 14, “Liposomes,” Drug Carriers in Biology and Medicine, pp. 287-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673 and 3,625,214, and Jein, TIPS 19:155-157 (1998), the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, osmotic minipumps are used to provide controlled sustained delivery of pharmaceutical compositions described herein, through cannulae to the site of interest, e.g. directly into a tissue at the site of metastatic growth or into the vascular supply of a tumor. The pump can be surgically implanted, for example continuous administration of endostatin, an anti-angiogenesis agent, by intraperitoneally implanted osmotic pump is described in Cancer Res. 2001 Oct. 15; 61(20):7669-74. Therapuetic amounts of RhoH cDNA expression vectors or RhoH cDNA can also be continually administered by a external pump attached to an intravenous needle.

In one embodiment, the pharmaceutical compositions are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

For enteral administration, a composition can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for oral administration may be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS(N,N-bis(3-D-gluconamidopropyl)-cholamide), chlorobutanol, octoxynol-9, benzyl alcohol, phenols, cresols, and alkyl alcohols. An especially preferred oral absorption enhancer for the present invention is sodium lauryl sulfate.

The route of administration, dosage form, and the effective amount vary according to the potency of the RhoH cDNA and the RhoH cDNA expression vectors, whether the vectors are viral or non-viral vectors, their physicochemical characteristics, and according to the treatment location. The selection of proper dosage is well within the skill of an ordinarily skilled physician. For example, dosage can be about 1 mg, about 50 mg, or about 100 mg. Equivalent dosages can also be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months.

In embodiment, periodic monitoring of the expression of the exogenous RhoH cDNA in the treated mammal is performed by qRT-PCT methods described herein.

In one embodiment, dosage forms include pharmaceutically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of compositions include polysaccharides such as sodium carboxymethylcellulose or methylcellulose, polyvinylpyrrolidone, polyacrylates, polyoxyethylene-polyoxypropylene-block polymers, polyethylene glycol and wood wax alcohols. For all administrations, conventional depot forms are suitably used. Such forms include, for example, microcapsules, nano-capsules, liposomes, plasters, inhalation forms, nose sprays, sublingual tablets, and sustained release preparations. For examples of sustained release compositions, see U.S. Pat. No. 3,773,919, EP 58,481A, U.S. Pat. No. 3,887,699, EP 158,277A, Canadian Patent No. 1176565, U. Sidman et al., Biopolymers 22:547 (1983) and R. Langer et al., Chem. Tech. 12:98 (1982). The RhoH cDNA or RhoH cDNA expression vector will usually be formulated in such vehicles at a concentration of about 0.1 mg/ml to 100 mg/ml and the viral vector should be in the range of 10⁶ to 1×10¹⁴ viral vector particles per application per patient.

In one embodiment, other ingredients may be added to pharmaceutical formulations, including antioxidants, e.g., ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

In one embodiment, the pharmaceutical formulation to be used for therapeutic administration must be sterile. Sterility is readily accomplished by filtration through sterile filtration membranes (e.g., 0.2 micron membranes). The RhoH cDNA or RhoH cDNA expression vector can be stored in lyophilized form or as an aqueous solution if it is highly stable to thermal and oxidative denaturation. The pH of the RhoH cDNA or RhoH cDNA expression vector preparations typically can be about from 6 to 8.

This invention is further illustrated by the following example which should not be construed as limiting. The contents of all references cited throughout this application, as well as the figures and table are incorporated herein by reference.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

### EXAMPLE

### Materials and Methods

Cell Culture—The hairy-cell line Mo, the T-cell lines CEM and HSB-2 and the B-cell lines Raji and U-266 were obtained from the American Type Culture Collection (ATCC) and grown according to their specifications⁴⁰⁻⁴⁵. The B-cell line VAL was kindly provided by Dr. Christian Bastard (Laboratoire de Génétique Oncologique, Centre Henri Becquerel, Rouen, France)⁴⁶. The hairy-cell line HC-1 was obtained from the Deutsche Sammlung von Mikroorganismen und Zellkulturen GmbH (DSMZ) and grown according to their specifications⁴⁷. The hairy-cell line EH was kindly provided by Dr. Guy B. Faguet (Veterans Administration Medical Center, Augusta, Ga.)⁴⁸. The hairy-cell line ESKOL was kindly provided by Edward F. Srour (Indiana University School of Medicine, Indianapolis, Ind.)⁴⁹. The hairy-cell lines JOK-1 and JC-1 were kindly provided by JØrn Koch (Aarhus University Hospital, Aarhus, Denmark)⁵⁰. Human microvascular endothelial cells (HMEC-1) were a kind gift of Dr. Sean P. Colgan (Brigham and Women's Hospital, Boston, Mass.)⁵¹. CdCl₂ was obtained from Fluka Chemie AG (Buchs, Switzerland) and used at concentrations of 1 μM or 10 μM where indicated. Lipopolysaccharide (LPS) was obtained from EMD Biosciences, Inc. (San Diego, Calif.) and used at a final concentration of 100 ng/ml where indicated.

Isolation of HCL and Normal B-Lymphocytes—Blood was drawn from normal volunteers and patients with HCL. Samples from patients with HCL were provided by Dr. Xavier Troussard (Laboratoire d'Hématologie, CHU de Caen, France) and samples from normal volunteers were provided by Dr. Christophe Roumier (Laboratoire d'Hématologie, Hôpital Roger Salengro, Lille, France). Leukocytes were prepared by ficoll-gradient centrifugation and frozen in liquid nitrogen. After thawing, cells were washed twice in RPMI 1640 containing 15% FBS, then resuspended in ice-cold PBS supplemented with 0.5% FBS and 2 mM EDTA. B-lymphocytes were isolated by negative selection using the B-Cell Isolation Kit II (Miltenyi Biotec, Paris, France). Cells isolated from HCL patients were determined to be over 90% positive for surface expression of CD19 and CD103 by flow cytometry. Prior to RNA isolation, B-lymphocytes were washed once in PBS supplemented with 0.5% FBS but lacking EDTA.

Plasmid Construction—The activity of the CD11c promoter was assessed using the expression vector pATLuc that contains a promoterless firefly luciferase reporter gene⁵². PCR was used to generate a fragment of the CD11c gene representing nucleotides −128 to +36 relative to the major transcription initiation site. This fragment was then subcloned into the “filled-in” HindIII site of pATLuc to generate p11c-Wt^{13, 53}. The ability of RhoH to repress the CD11c promoter was assessed using the expression constructs pCMV-RhoH and pMEP-RhoH. These were generated by first isolating the EcoRI/XbaI and KpnI/XbaI fragments of pcDNA3-VSVG-RhoH-Wt that encode RhoH tagged at its N-terminus with the Vesicular Stomatitis Virus Glycoprotein (VSVG) epitope. The isolated EcoRI/XbaI fragment was then cloned between the EcoRI and XbaI sites of pcDNA3.1Zeo(+) (Invitrogen Corp., Carlsbad, Calif.) to produce pCMV-RhoH. The KpnI/XbaI fragment containing RhoH was cloned between the KpnI and NheI sites of pMEP4 (Invitrogen Corp.) to generate pMEP-RhoH. The transfection control plasmid pRSV-βwas purchased from Promega Corp. (Madison, Wis.).

RhoH protomer construct and assay of promoter activity—A fragment of the RhoH gene representing nucleotides −236 to +67 relative to a transcription initiation site used in B-lymphocytes was cloned upstream of the luciferase gene in pGL4.14[Luc2/Hygro] to generate the construct pGL4-RhoH. The transfection control plasmid pSV-βGal was mixed with either the parental vector pGL4.14[Luc2/Hygro] or pGL4-RhoH. The two plasmid mixtures were then transfected into the HCL B-lymphocyte cell-line EH or the non-HCL B-lymphocyte cell-line Raji. The levels of β-galactosidase activity directed by pSV-βGal were taken as reflective of transfection efficiency and used to correct the luciferase assay results. After correction for transfection efficiency, the level of luciferase reporter gene activity directed by pGL4-RhoH above that conferred by pGL4.14[Luc2/Hygro] was calculated.

RNA Isolation—Total RNA was isolated from cell lines and preparations of human blood cells by use of the High Pure RNA Isolation Kit (Roche Diagnostics, Meylan, France). The procedure included a DNAse I treatment to eliminate genomic DNA contamination. Amounts and quality of RNA were evaluated by UV spectroscopy using an Agilent 2100 Bioanalyser (Agilent Technologies, Massy, France).

Polymerase Chain Reactions (PCR)—The cDNA templates for quantitative-reverse transcriptase-PCR (Q-RT-PCR) were prepared by random priming of 5 μg of total RNA with 200 units of M-MLV reverse transcriptase in a final volume of 100 μl. The quality and quantity of templates was evaluated by analysis of ABL mRNA in accordance with the recommendations of Europe Against Cancer⁵⁴. The B-cell line Namalwa that expresses high levels of ABL and RhoH mRNA was used to produce calibration curves that evaluated PCR efficiency. RhoH and ABL mRNA were measured separately using TaqMan technology (Applied Biosystems, Foster City, Calif.). Real time quantitative PCR primers pairs and probes were chosen with the assistance of the computer program Primer Express (Applied Biosystems). Due to heterogeneity at the 5′ end of RhoH mRNA, care was taken to design PCR primers that amplified all RhoH transcripts⁵⁵. The sense primer used had the nucleotide sequence 5′-TTTGGAAACTTCTCCTTCACACAC-3′ (SEQ. ID No. 3) and the anti-sense primer had the sequence 5′-GCCCATCCAAGCACCGT-3′ (SEQ. ID No. 4). The size of the PCR product resulting from the use of these primers was 171 bp and, therefore, compatible with good amplification efficiency. The RhoH probe had the sequence 5′-AGTTGAAGACTAGGCTTT-3′ (SEQ. ID No. 5) and was labeled at its 5′-end with 6-carboxy-fluorescein phosphoramidite (FAM) and at its 3′-end with non-fluorescent quencher (NFQ). The ABL probe that was used as control for cDNA quantity and quality was also labeled with FAM at its 5′-end but with 6-carboxy-tetramethyl-rhodamine (TAMRA) as quencher at its 3′-end. Each PCR reaction was performed in a 25 μl total volume, using 12.5 μl of 2×UDG Master mix (Applied Biosystems), 10 μM each of sense and anti-sense primer and approximately 100 ng of cDNA template. Each PCR run included samples containing no cDNA and Namalwa cDNA that produced calibration curves for ABL and RhoH gene expression. The relative level of endogenous RhoH expression (E-RhoH) was calculated by dividing the quantitative level of RhoH expression (Q-RhoH) by the quantitative level of ABL expression (Q-ABL). All relative levels of RhoH expression were determined from two independent preparations of RNA each of which was used in two independent cDNA synthesis reactions. Each cDNA synthesis was then analyzed by two independent quantitations performed in duplicate. Expression of recombinant RhoH tagged with the VSVG epitope was evaluated by RT-PCR using a sense primer with the nucleotide sequence 5′-ATGGGTTACACCGACATCGAGATGACC-3′ (SEQ. ID No. 8) and an anti-sense primer with the nucleotide sequence 5′-TCTCGTACACTGTGGGCTTGTAGGC-3′ (SEQ. ID No. 9). This primer pair generates PCR products only from transcripts that encode VSVG-RhoH and not from transcripts that originate from the endogenous RhoH gene.

Transfection—EH and Mo cells were transfected by electroporation using 1 μg of pRSV-β and 16 μg of pCMV-RhoH mixed with either 8 μg of pATLuc or 8 μg of p11c-Wt. As controls for these experiments parallel transfections were performed with the empty parental vector pCMV from which pCMV-RhoH was derived. JOK-1 cells were transfected in the same way as EH and Mo except that pCMV-RhoH was replaced with pMEP-RhoH and pCMV was replaced with pMEP4. In addition, the culture medium to which transfected JOK-1 cells were placed contained 1 μM CdCl₂ to drive RhoH expression from the metallothionine promoter present in pMEP4. In all transfection experiments, sixteen hours after electroporation cells were processed for assay of β-galactosidase and luciferase activity.

Generation of Stable Cell Line Pools—The plasmids pMEP4 and pMEP-RhoH were linearized by digestion with ClaI then transfected by electroporation into JOK-1 cells. Pools of JOK-1 cells that contained the transfected plasmids stably integrated within there genome were selected by treatment with 150 μg/ml of hygromycin.

Flow Cytometric Analysis—Flow cytometry was performed by incubating 5×10⁵ cells with a PE-conjugated version of the monoclonal antibody BU15 directed against CD11c (Beckman-Coulter, Paris, France). The isotype-matched control for these experiments utilized a PE-conjugated version of the IgG1 clone 679-Mc7 (Beckman-Coulter, Paris, France). After staining, cells were fixed with 1% paraformaldehyde and analyzed using an EPICS XL-MCL flow cytometer (Beckman-Coulter, Paris, France). Phenotypic evaluation of B-cells isolated from HCL patients was performed by FACS analysis after double-labeling with anti-CD19 and anti-CD103 antibodies conjugated, respectively with PE and FITC (Beckman-Coulter, Paris, France). Phenotypic evaluation of B-cells isolated from normal volunteers was performed by FACS after labeling with the anti-CD19 antibody.

Homotypic Adhesion Assays—Petri dishes were seeded with 5×10⁵ cells per ml and incubated for three days. Representative fields of the cultures were photographed after one and three days using an inverted phase contrast microscope (DMIL, Leica, Rueil-Malmaison, France) equipped with a video camera and Leica Q-fluoro standard Image Software. In parallel, the number and size of the aggregates in the cultures were evaluated using a Leica 52 7001 ocular objective equipped with a micrometric grid of 1 mm divided into 100 squares of 10⁴ μm². All measurements represented the average values obtained from six representative fields. The size of the aggregates was evaluated as a function of time by measurement of their surface area expressed in μm². Aggregates were classified into the three size categories of “small” (S: 0.1-0.25×10⁴ μm²), “medium” (M: 0.5-1.0×10⁴ μm²) and “large” (L: 1.5-20.0×10⁴ μm²). The percentage of aggregates in each category was then calculated using the formula: (Number of clumps in a given category/Total number of clumps)×100. The percentage of cells that were not engaged in aggregates was also analyzed. In this case measurements were averaged from 10 representative fields and obtained by counting every cell not engaged in an aggregate. The percentage of non-aggregated cells per field was then calculated using the formula: (Number of non-aggregated cells/Total number of cells)×100. The total number of cells per field was determined by counting all cells after disruption of the aggregates in the entire cultures and calculated by the formula: Total number of cells in the cultures/R, where R represents the ratio of the surface areas of the culture dish and the micrometric grid. Specifically, with our conditions of culture, the culture dish had an area of 50 cm² and the micrometric grid an area of 1 mm². Therefore, R was equal to 5000.

Lymphocyte-Endothelial Adhesion Assays—Monolayers of the endothelial cell line HMEC-1 were seeded into 96 multiwell culture plates and confluency checked after 48 hours by staining with Crystal Violet. Monolayers were either left untreated or activated for 18 hours with 100 ng/ml of lipopolysaccharide (LPS). Pools of JOK-1 cells stably transfected with pMEP4 or pMEP-RhoH cells were incubated for 30 minutes at 37° C. in the presence of 5 μM BCECF-AM (Calbiochem, EMD Biosciences Inc., San Diego, Calif.) then washed three times in Hanks Balanced Salt Solution (HBSS). 10⁵ labeled cells were added to each well containing activated or non-activated monolayers, culture plates were then centrifuged for 4 minutes at 150 g to effect uniform settling and adhesion allowed for one hour at 37° C. Monolayers were then gently washed three times in HBSS and fluorescence intensity measured at 485 nm using a Mithras LB 940 multimode reader (Berthold Technologies, GmbH & Co KG, Bad Wildbad, Germany). All values were normalized by subtraction of the fluorescence intensity of monolayers incubated in buffer alone. Specific cell adhesion was assessed by subtracting the fluorescence intensities obtained from non-activated monolayers from the intensities obtained using activated monolayers. Adhesion assays for each stable JOK-1 pool were performed a total of three times at minimum in quadruplicate.

Trans-Endothelial Migration Assays—Confluent monolayers of HMEC-1 were prepared in the same way as for leukocyte-endothelial adhesion assays but in Transwell migration chambers of 6.5 mm diameter and 5.0 μm pore size (Corning Inc., Corning, N.Y.). Transwell inserts were set in HMEC-1 medium carried in 24 well plates and either left untreated or activated for 18 hours with 100 ng/ml of LPS. Next 10⁵ JOK-1 cells either stably expressing pMEP4 or pMEP-RhoH were prepared in 150 μl of HMEC-1 medium without LPS and added to the monolayers inside the Transwell inserts. These inserts were again set into 24 well plates and incubated for 8 hours. After this time the HMEC-1 medium under the Transwell inserts was supplemented with 100 ng/ml LPS to act as a chemoattractant. The Transwell inserts were then incubated at 37° C. for 16 hours and the JOK-1 cells that migrated through to the medium on their underside collected and counted. Migration assays were performed in triplicate a total of three times for each stable JOK-1 pool cultured with either activated or quiescent HMEC-1.

Xenograft Mouse Model—Male NOD-SCID mice (CB17 PRKDC, Charles River Laboratories, Saint Germain Sur L'Arbresle, France) aged seven weeks were injected intraperitonealy with 10⁷ JOK-1 cells suspended in RPMI 1640 medium without FBS. The JOK-1 cells that were injected either stably expressed pMEP-RhoH or the Empty vector, pMEP. Control mice were injected with the RPMI1640 vehicle alone. During the next four weeks the health of the mice was monitored and if death occurred analysis was performed immediately. After four weeks, surviving animals were sacrificed and their peritoneal cavity opened for tumor analysis. When present, tumors were dissected, their size measured and photographs taken. Two tumors were analyzed by cytometry to confirm they consisted of JOK-1 cells expressing human CD19.

### Example 1

### RhoH is Specifically Under-Expressed in Cell Lines Derived from HCL

Previous studies have demonstrated that the Ras family of proto-oncogenes is abnormally active in HCL^{13, 35}. Since RhoH represents a natural inhibitor of Ras in the hematopoietic lineage, the possibility that HCL may be characterized by RhoH under-expression was investigated. To address this question, quantitative real-time polymerase chain reactions (Q-RT-PCR) was used to examine RhoH mRNA levels in a range of cell lines derived both from patients with HCL and other forms of leukemia.

Total RNA was extracted from the HCL cell lines Mo, EH, JOK-1, ESKOL, JC-1 and HC-1. Total RNA was also extracted from the cell lines CEM and HSB-2 representing T-acute lymphoblastic leukemia, the cell line Raji representing Burkitt's lymphoma, U-266 representing plasmocytoma and VAL representing B-cell non-Hodgkin's lymphoma. The quantitative levels of RhoH and ABL expression were determined for each individual cell line by Q-RT-PCR. The relative level of RhoH expression (E-RhoH) was then calculated by dividing the quantitative level of RhoH expression (Q-RhoH) by the quantitative level of ABL expression (Q-ABL) 54.

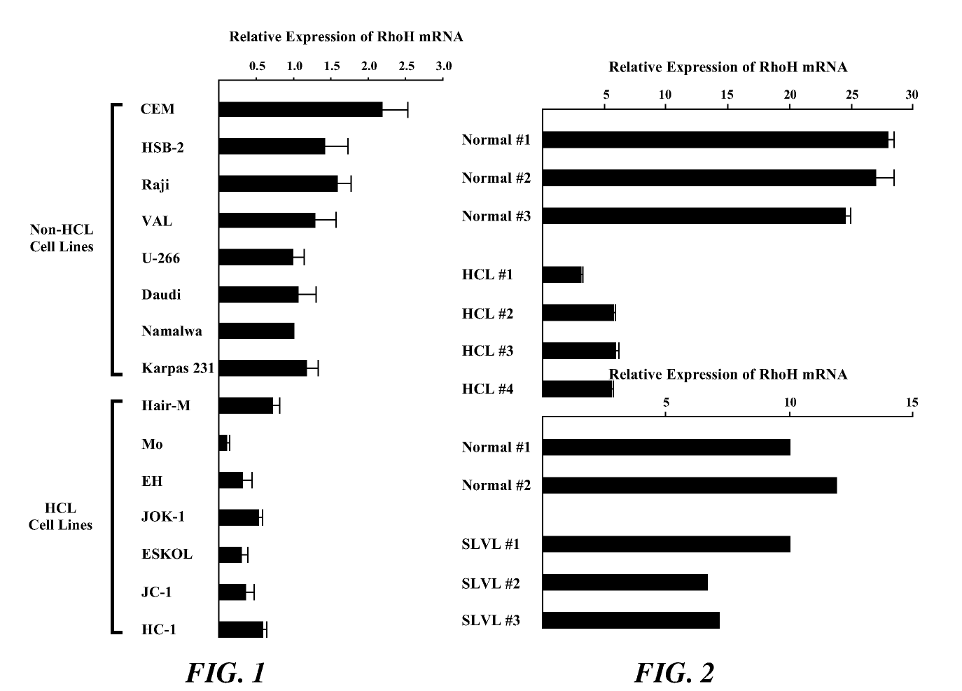

This study indicated that, on average, the relative expression of RhoH in HCL cell lines was 4.4 fold lower than that in cell lines derived from Burkitt's lymphoma (Raji), plasmocytoma (U-266), T-acute lymphoblastic leukemia (HSB-2 and CEM) and B-cell non-Hodgkin's lymphoma (VAL) (FIG. 1).

Total protein was also extracted from the HCL B-cell lines JOK-1, EH, HC-1 and Hair-M and from the non-HCL B-cell lines Raji and Namalwa. 50 μg of EH protein, 100 μg of Namalwa, Raji, JOK-1 and Hair-M protein and 150 μg of HC-1 protein were subject to SDS-PAGE followed by western blotting. RhoH protein expression was analyzed using the rabbit polyclonal anti-RhoH antibody α-Rho-6005. Protein loading was checked by hybridizing the blots with an anti-tubulin antibody. HCL B-cell lines has very low or undetectable RhoH protein and mRNA (data not shown).

### Example 2

### RhoH is Under-Expressed in Neoplastic Lymphocytes Isolated Directly from HCL Patients

Since RhoH appears under-expressed in HCL cell lines, the level of RhoH expression was also examined in malignant cells circulating within the blood stream of HCL patients. B-lymphocytes were isolated from the blood of three normal donors and from four patients with HCL. Total RNA was extracted from the B-lymphocytes of three normal individuals and from four patients with HCL. The relative levels of RhoH expression were then determined by Q-RT-PCR in the same way as for hematopoietic cell lines (FIG. 1). Examination by Q-RT-PCR demonstrated that RhoH expression levels were, on average, 5.2 lower in the B-lymphocytes of HCL patients compared to normal individuals (FIG. 2). In addition, B-lymphocytes were isolated from the normal spleens of two patients with ITP and the pathologic spleens of three patients with SLVL. The relative levels of expression of RhoH mRNA were determined and found to be in contrast to the substantial suppression of RhoH in HCL lymphocytes, splenic B-lymphocytes isolated from patients with SLVL exhibited, on average, only a 27% reduction in RhoH expression

### Example 3

### RhoH Under-Expression in HCL Cells is Mediated by Transcriptional Repression of the RhoH Gene Promoter

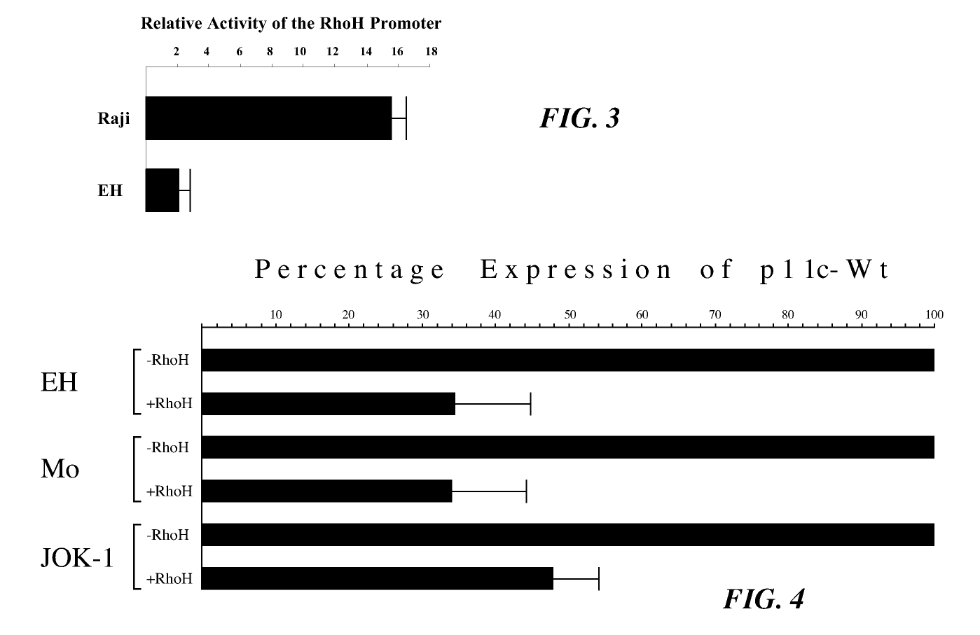

RhoH under-expression in HCL could be mediated by transcriptional and/or post-transcriptional events. The former possibility was investigated by isolating the human RhoH promoter spanning nucleotides −236 to +67 relative to a transcription initiation site utilized in B-lymphocytes (46). This promoter region was then cloned upstream of the luciferase reporter gene present in pGL4.14[Luc2/Hygro] to generate the construct pGL4-RhoH. When pGL4-RhoH was transfected into EH hairy-cells it directed expression only 2.1 fold above that directed by pGL4.14[Luc2/Hygro] empty of RhoH gene sequences (FIG. 3). In contrast, when pGL4-RhoH was transfected into the non-hairy cell B-lymphocyte cell line Raji it directed expression 15.6 fold above background (FIG. 3). Thus the transcriptional activity of the wild-type RhoH promoter is 7.4 fold lower in hairy-cell leukemia cells than in non-hairy cells. These results indicate that transcriptional repression of the RhoH gene promoter plays a major role in RhoH under-expression in hairy-cell leukemia cells.

### Example 4

### Transient Reconstitution of Rhoh Expression Represses the Aberrant Activity of the CD11c Gene Promoter that Characterizes HCL

The β2-integrin CD11c is normally largely restricted in its expression pattern to cells of the myeloid lineage¹¹. Its abnormal expression on the surface of HCL lymphocytes is used as a diagnostic marker for the disease⁹. Previous studies have shown that HCL abnormal expression of the CD11c protein reflects abnormal transcription of the gene by which it is encoded¹³. This abnormal transcription is Ras-dependent and driven by the promoter region of the CD11c gene extending from −128 to +36 relative to the major site of transcription initiation. Since high levels of Ras-dependent transcription from the CD11c promoter are characteristic of HCL, the currently identified under-expression of RhoH might be the underlying cause. In order to test this possibility, the HCL cell lines Mo, EH and JOK-1 were employed.

The activity of the CD11c promoter was assessed using the expression vector pATLuc that contains a promoterless firefly luciferase reporter gene⁵². A fragment of the CD11c gene representing nucleotides −128 to +36 relative to the major transcription initiation site was cloned upstream of the luciferase gene in pATLuc to generate p11c-Wt 13,⁵³. EH, Mo and JOK-1 cells were transfected in parallel with p11c-Wt and pATLuc each mixed with the plasmid pRSV-β that constitutively expresses β-galactosidase. Transfections of EH and Mo were performed in the presence of pCMV-RhoH, that constitutively expresses RhoH, or in the presence of its empty equivalent pCMV. Transfections of JOK-1 were performed in the presence pMEP-RhoH, where RhoH is expressed from the metallothionine promoter, or the empty parental plasmid pMEP4. Transfected JOK-1 cells were cultured with 1 μM CdCl2 and transfected EH and Mo were cultured without CdCl2 for 16 hours before β-galactosidease and luciferase assays were performed. The levels of β-galactosidase activity were taken as reflective of transfection efficiency and used to correct the firefly luciferase assay results. After correction for transfection efficiency, the level of luciferase reporter gene activity directed by p11c-Wt above that conferred by pATLuc in the presence of pCMV or pMEP4 was assigned a value of 100% (-RhoH). The level of activity directed by p11c-Wt in parallel transfections in the presence of pCMV-RhoH or pMEP-RhoH is expressed as a percentage of this value (+RhoH). The construct constitutively expressing human RhoH was transfected into these cell lines in combination with either a CD11c promoter construct or its parental vector empty of CD11c sequences. In Mo, EH and JOK-1 the expression of exogenous RhoH caused, on average, 66%, 66% and 52% reductions, respectively, in the transcriptional activity of the CD11c promoter (FIG. 4). These results demonstrated that RhoH repression contributes to the expression of the CD11c promoter in hairy-cells.

### Example 5

### Stable Reconstitution of RhoH Expression Reduces Cell-Surface Expression of CD11c Protein

In JOK-1 hairy-cells transient reconstitution of RhoH expression represses the CD11c gene promoter when the promoter is present in an extrachromosomal plasmid (FIG. 4). Next the possibility that RhoH reconstitution might also cause a reduction in the surface expression of the CD11c protein was investigated as this would reflect repression of the endogenous CD11c gene.

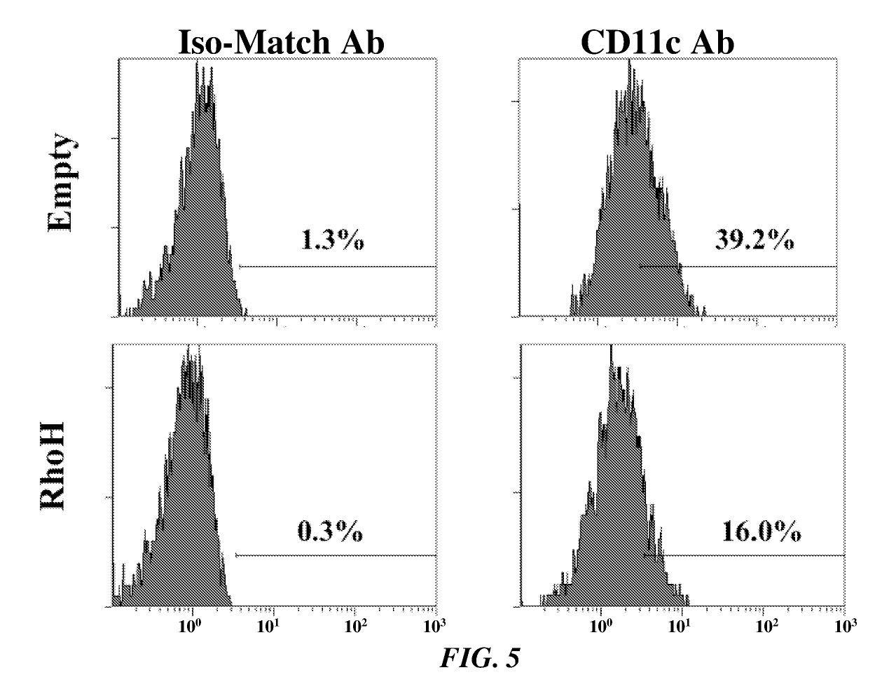

Pools of JOK-1 cells were generated that contain within their genome either pMEP4 (an empty expression plasmid) or pMEP-RhoH (the same plasmid constitutively expressing RhoH). Total RNA was isolated from these cells and the constitutively expressing RhoH mRNA was detected by RT-PCR. Agarose gel was stained with ethidium bromide following electrophoresis of RT-PCR products generated from JOK-1 cells expressing pMEP4 or pMEP-RhoH. The pairs of oligonucleotides used in the PCR generated products from mRNA encoding recombinant RhoH tagged with the VSVG epitope (RhoH) or from the ABL gene product (ABL). The oligonucleotides fail to generate products from endogenous RhoH mRNA (data not shown). This analysis demonstrated that cells carrying pMEP4 expressed no-recombinant RhoH while cells carrying pMEP-RhoH produced recombinant RhoH either in the presence or absence of supplemental CdCl₂. Flow cytometric analysis was performed on JOK-1 stably expressing pMEP4 (Empty) and on JOK-1 cells stably expressing pMEP-RhoH (RhoH). Both cell lines were cultured in the absence of supplemental CdCl₂ and cytometry was performed using a monoclonal antibody that specifically binds CD11c (CD11c Ab) or an isotype matched control (Iso-Match Ab). The percentages of cells that exhibit fluorescence in a defined range of intensity are presented from a representative of three independent experiments. The surface expression of CD11c in these two pools of cells was then assessed using flow cytometry. This analysis established that stable reconstitution of RhoH expression causes, on average, a 49.5% decrease in CD11c expression (FIG. 5). Consequently, in JOK-1 cells reconstitution inhibits endogenous as well as exogenous CD11c expression.

### Example 6

### Reconstitution of RhoH Expression Inhibits the Homotypic Adhesion of HCL

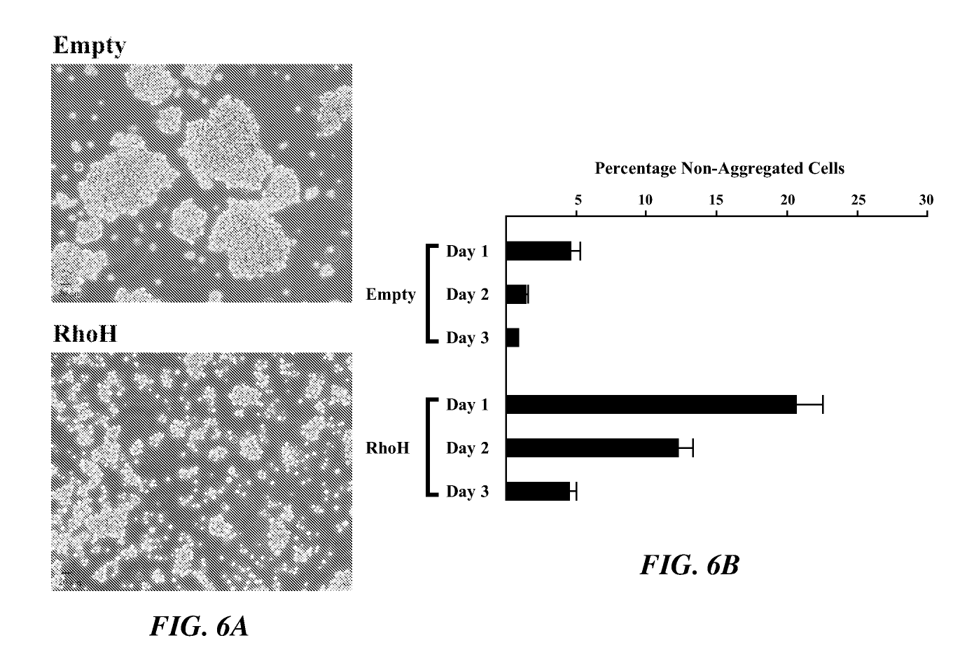

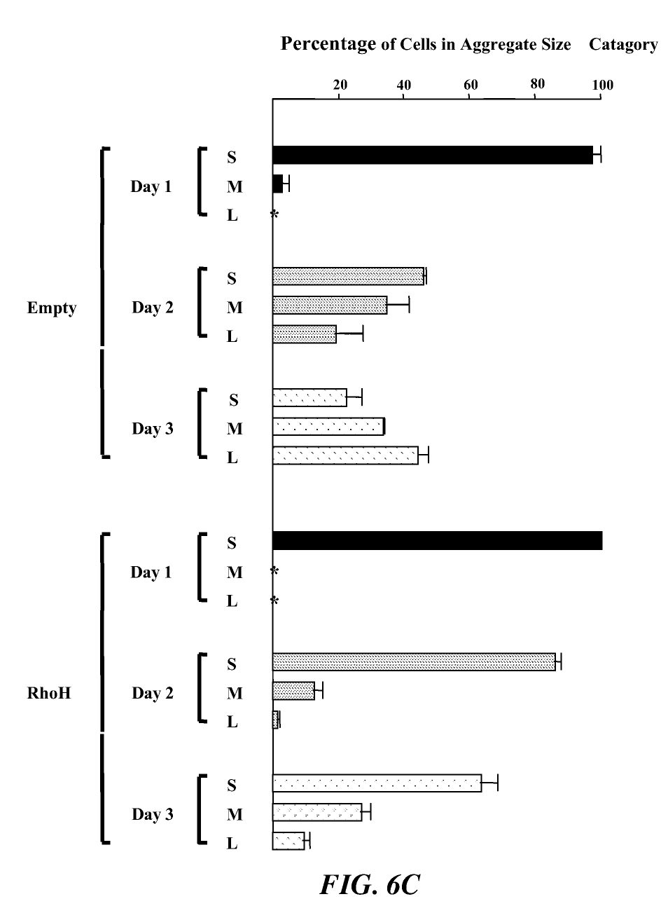

One of the hallmarks of HCL is the extravasation of neoplastic lymphocytes^{1, 3}. Such extravasation is dependent upon malignant cells exhibiting increased inter-cellular adhesion. In cell culture this increased capacity for adhesion is manifest by HCL cell lines typically growing in large clumps. It was have demonstrated that RhoH reconstitution in HCL reduces expression of the pro-adhesion molecule CD11c. Others have shown that RhoH holds the CD11a/CD18 heterodimer in an inactive conformation⁵⁶. Since RhoH appears to inhibit functional expression of molecules that mediate leukocyte adhesion, the hypothesis that RhoH reconstitution in HCL might inhibit homotypic inter-cellular adhesion was tested. The pools of JOK-1 that either stably express RhoH or an empty vector were disrupted into cultures of single cells and then incubated undisturbed for three days. FIG. 6A shows the light microscopy of these cells after 3 days. After this period of time, it was found that, compared to the pools expressing the empty vector, the pools expressing RhoH had over 5 fold fewer cells growing as aggregates (FIG. 6B). In addition, the sizes of the aggregates that did exist were significantly reduced (FIG. 6C).

### Example 7

### Reconstitution of RhoH Expression Inhibits the Heterotypic Adhesion of HCL

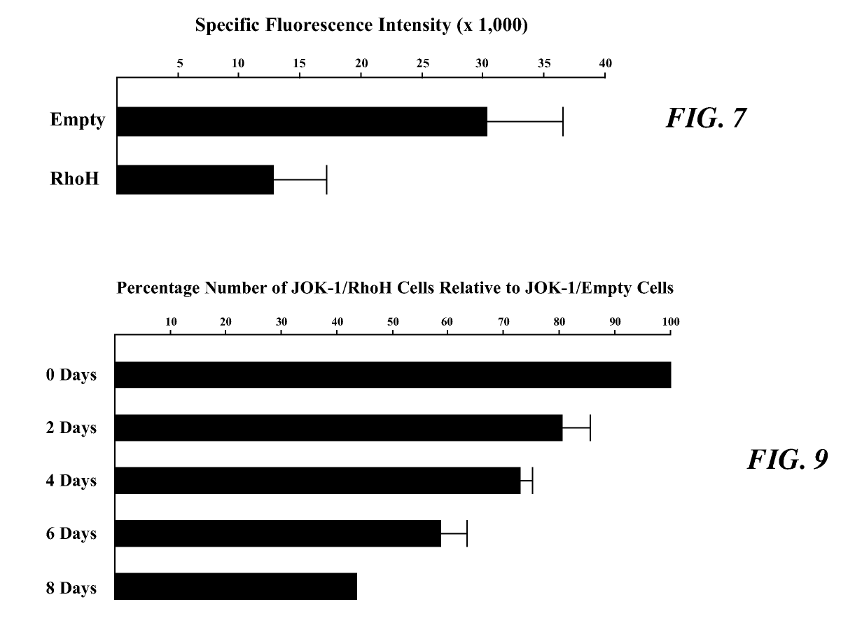

The study of aggregation indicated that RhoH reconstitution in HCL cells inhibits their homotypic adhesion (FIG. 6). However, central to the process of HCL extravasation is the heterotypic adhesion of malignant cells to vascular endothelium. The ability of RhoH reconstitution to inhibit this kind of adhesion was assessed in vitro using a fluorescence assay that was previously employed⁵⁷. Pools of JOK-1 cells stably transfected with pMEP4 (Empty) or pMEP-RhoH (RhoH) were cultured in the absence of supplemental CdCl₂ and labeled with the fluorescent marker BCECF. Cells were then incubated for one hour with monolayers of HMEC-1 activated or untreated with LPS. Monolayers were next gently washed and fluorescence intensity measured at 485 nm. All values were normalized by subtraction of the fluorescence intensity of monolayers incubated in buffer alone. Specific fluorescence intensity was then determined by subtracting the fluorescence intensity obtained from non-activated monolayers from that obtained using monolayers activated with LPS. Use of this assay demonstrated that the pools of JOK-1 hairy-cells reconstituted with RhoH exhibit a 58% reduction in adhesion to activated endothelial cells compared to control pools (FIG. 7).

### Example 8

### Reconstitution of RhoH Expression Inhibits HCL Trans-Endothelial Migration

In addition to simple endothelial adhesion, HCL extravasation is dependent upon malignant cells being able to migrate through endothelium. Consequently, Transwell migration chambers was used to assess the ability of RhoH reconstitution to inhibit HCL trans-endothelial migration.

JOK-1 cells stably transfected with pMEP4 (Empty) or pMEP-RhoH (RhoH) were cultured in the absence of supplemental CdCl₂ then settled on top of quiescent (-LPS) or activated (+LPS) monolayers of HMEC-1 within Transwell migration chambers. After 8 hours the culture medium present below the HMEC-1 monolayers was supplemented with 100 ng/ml LPS to act as a chemoattractant for the JOK-1 cells. 16 hours later the JOK-1 cells that migrated through the support membrane of the endothelial monolayer were collected and counted.

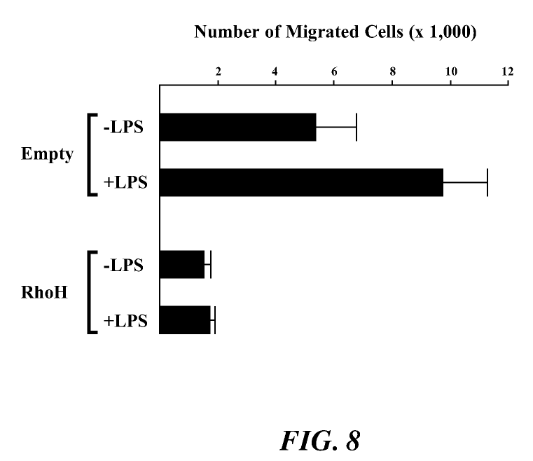

Equal numbers of control JOK-1 pools and pools reconstituted with RhoH were settled for 8 hours on top of quiescent or activated endothelial monolayers. JOK-1 migration was then elicited by adding LPS to the culture medium present below the monolayers. After 16 hours the JOK-1 cells that had migrated both through the endothelial monolayer as well as its support membrane were collected and counted. With quiescent endothelial monolayers, JOK-1 cells reconstituted with RhoH exhibited a 3.5 fold reduction in migration capacity compared to control cells. When endothelial monolayers were activated, the degree of reduction reached 5.5 fold (FIG. 8). This more pronounced reduction was caused by endothelial activation inducing the migration of control cells by 81% but inducing the migration of cells reconstituted with RhoH by only 16%.

### Example 9

### Reconstitution of RhoH Expression Reduces HCL Proliferation

Abnormal inter-cellular adhesion that effects trans-endothelial migration represents one of the pathologic hallmarks of HCL. The finding that such processes are dependent upon under-expression of RhoH suggests that reconstitution of RhoH may represent a useful therapeutic strategy. This possibility was further assessed by determining the ability of RhoH reconstitution to block HCL proliferation. Equal numbers of JOK-1 cells containing pMEP4 or pMEP4-RhoH were cultured in parallel and after 0, 2, 4, 6 and 8 days the cells were counted. The growth medium used in these experiments did not contain supplemental CdCl₂. The number of cells containing the empty vector pMEP4 was assigned a value of 100% and the number of cells containing the RhoH plasmid calculated as a percentage of this value. Histograms represent the mean of two independent experiments.

Cultures were initiated from equal numbers of the JOK-1 pools that contained stable integration of an empty vector or this same vector expressing RhoH. The two pools were cultured in parallel and after 0, 2, 4, 6 and 8 days the cells were counted. The number of cells containing the empty vector was assigned a value of 100% and the number of cells containing the RhoH plasmid calculated as a percentage of this value. The results depicted in FIG. 9 indicate that RhoH reconstitution causes a reduction in HCL proliferation such that after 8 days the number of JOK-1 cells was reduced, on average, by 57%.

### Example 10

### RhoH Reconstitution Protects Against Malignant Progression In Vivo

The data generated in vitro demonstrates that RhoH reconstitution inhibits both the proliferation and adhesion of HCL cells. A xenograft mouse model was utilized to determine whether RhoH reconstitution inhibits malignant progression in vivo.

The peritoneum of SCID mice was injected with RPMI1640, RPMI1640 containing JOK-1 cells stably expressing the empty vector pMEP or RPMI1640 containing JOK-1 cells stably expressing the RhoH expression construct pMEP-RhoH. After 28 days all surviving mice were sacrificed and the injection site and/or spleens examined.

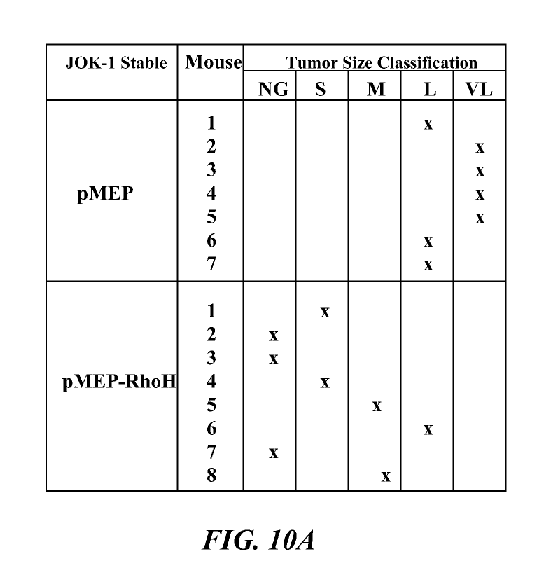

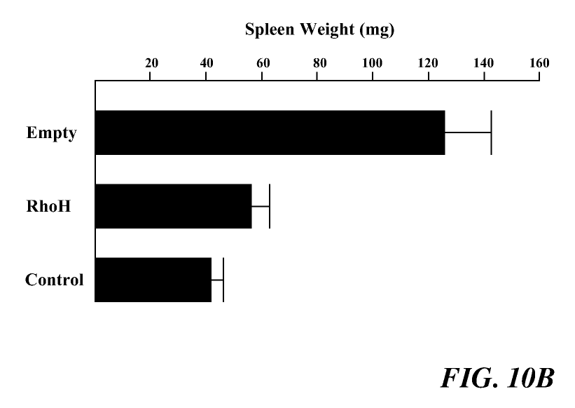

The JOK-1 pools that contained stable integration of an empty vector or this same vector expressing RhoH were injected into the peritoneum of NOD-SCID mice. Twelve mice were injected with each pool. During the course of 28 days all mice injected with cells reconstituted with RhoH remained alive and vigorous. In contrast, all mice injected with non-reconstituted cells became increasingly sluggish with three dying after 27 days and another dying a day later. At this time all surviving animals, including the vigorous subjects injected with RhoH reconstituted cells were sacrificed. The peritoneal cavity was opened and the site of injection examined in seven recipients of non-reconstituted cells and eight recipients of reconstituted cells. They were classified depending upon whether they had no visible tumor (NG), a “small” tumor of less than 1 cm in maximum diameter (S), a “medium” sized tumor of 1-1.4 cm (M), a “large” tumor of 1.5-2.4 cm (L) or a “very large” tumor of 2.5-3.5 cm (VL). In the eight examined recipients of HCL cells reconstituted with RhoH, three failed to develop any manifestation of malignancy, two developed tumors of less than one centimeter in maximum dimension and the other three had tumors of maximum dimension 1.2, 1.4 and 1.8 cm. These observations were in striking contrast to mice injected with HCL cells not reconstituted with RhoH expression. Here the seven mice with non-reconstituted cells examined all developed tumors of at least 2.1 cm maximum dimension and two had tumors of a maximum dimension over 3 cm (FIG. 10A).

The weight of the spleen of these mice were also determined. Weight of the spleens isolated from mice injected with RPMI1640 alone (Control) or mice injected with JOK-1 cells expressing either pMEP (Empty) or pMEP-RhoH (RhoH). Histograms depict the mean weight +S.E.M. of four, nine and twelve mice, respectively. The spleens of mice with non-reconstituted cells were twice as large as those of mice with RhoH reconstituted cells (FIG. 10B). Taken together, this data generated using mice indicates that reconstituting RhoH expression inhibits malignant progression in vivo.

The references cited herein and throughout the specification are incorporated herein by reference.

### REFERENCES

1 Bouroncle B A, Wiseman B K, Doan C A. Leukemic reticuloendotheliosis. Blood. 1958; 13:609-630.
2 Allsup D J, Cawley J C. Diagnosis, biology and treatment of hairy-cell leukaemia. Clin. Exp. Med. 2004; 4:132-138.
3 Platanias L C, Golomb H M. Hairy cell leukaemia. Baillieres Clin. Haematol. 1993; 6:887898.
4 Robak T. Current treatment options in hairy cell leukemia and hairy cell leukemia variant. Cancer Treat Rev. 2006; 32:365-376.
5 Mey U, Strehl J, Gorschluter M, Ziske C, Glasmacher A, Pralle H, Schmidt-Wolf I. Advances in the treatment of hairy-cell leukaemia. Lancet Oncol. 2003; 4:86-94.
6 Zypchen L N, Nantel S H, Barnett J, Noble M, Gascoyne R D, Connors J M. Cladribine does not cure hairy cell leukemia (HCL). Blood. 2003; ASH Abstract 1593.
7 Golde D W, Stevens R H, Quan S G, Saxon A. Immunoglobulin synthesis in hairy cell leukemia. Brit. J. Haematol. 1977; 35:359-365.
8 Saxon A, Stevens R H, Golde D W. T-lymphocyte varient of hairy-cell leukemia. Ann. Int. Med. 1978; 88:323-326.
9 Schwarting R, Stein H, Wang C Y. The monoclonal antibodies αS-HCL1 (αLeu-14) and αS-HCL3 (αLeu-M5) allow the diagnosis of hairy cell leukemia. Blood. 1985; 65:974-983.
10 Yam L T, Li C Y, Lam K W. Tartrate-resistant acid phosphatase isoenzyme in the reticulum cells of leukemic reticuloendotheliosis. N. Engl. J. Med. 1971; 284:357-360.
11 Amaout M A. Structure and function of the leukocyte adhesion molecules CD11/CD18. Blood. 1990; 75:1037-1050.
12 Lam K W, Li O, Li C Y, Yam L T. Biochemical properties of human prostatic acid phosphatase. Clin. Chem. 1973; 19:483-487.
13 Nicolaou F, Teodoridis J M, Park H, Georgakis A, Farokhzad O C, Bottinger E P, Da Silva N, Rousselot P, Chomienne C, Ferenczi K, Arnaout M A, Shelley C S. CD11c Gene Expression in Hairy-Cell Leukemia is Dependent Upon Activation of the Proto-Oncogenes ras and junD. Blood. 2003; 101:4033-4041.
14 Shaulian E, Karin M. AP-1 in cell proliferation and survival. Oncogene. 2001; 20:2390-2400.
15 Abate C, Patel L, Rauscher III F J, Curran T. Redox regulation of fos and jun DNA-binding activity in vitro. Science. 1990; 249:1157-1161.
16 Boyle W J, Smeal T, Defize L H K, et al. Activation of protein kinase C decreases phosphorylation of c-jun at sites that negatively regulate its DNA-binding activity. Cell. 1991; 64:573-584.
17 Angel P, Imagawa M, Chiu R. et al. Phorbol ester-inducible genes contain a common cis element recognized by a TPA-modulated trans-acting factor. Cell. 1987; 49:729-739.
18 Lamph W W, Wamsley P, Sassone-Corsi P, Verma I M. Induction of proto-oncogene JUN/Ap-1 by serum and TPA. Nature. 1988; 334:629-631.
19 Angel P, Hattori K, Smeal T, Karin M. The jun proto-oncogene is positively autoregulated by its product, Jun/Ap-1. Cell. 1988; 55:875-885.
20 Wilson T, Treisman R. Removal of poly(A) and consequent degradation of c-fos mRNA facilitated by 3′ AU-rich sequences. Nature. 1988; 336:396-399.
21 Nakamura T, Datta R, Kharbanda S, Kufe D. Regulation of jun and fos gene expression in human monocytes by the macrophage colony-stimulating factor. Cell Growth Differ. 1991; 2:267-272.
22 Abate C, Luk D, Curran T. A ubiquitous nuclear protein stimulates the DNA-binding activity of fos and jun indirectly. Cell Growth Differ. 1990; 1:455-462.
23 Abate C, Luk D, Gentz R, Rauscher III F J, Curran T. Expression and purification of the leucine zipper and DNA-binding domains of Fos and Jun:both Fos and Jun contact DNA directly. Proc. Natl. Acad. Sci. USA. 1990; 87:1032-1036.
24 Xanthoudakis S, Curran T. Identification and characterization of Ref-1, a nuclear protein that facilitates AP-1 DNA-binding activity. EMBO J. 1992; 11:653-665.
25 Curran T, Morgan J I. Superinduction of c-fos by nerve growth factor in the presence of peripherally active benzodiazepines. Science. 1985; 229:1265-1268.
26 Barber J R, Verma I M. Modification of fos proteins: phosphorylation of c-fos, but not v-fos, is stimulated by 12-tetradecanoyl-phorbol-13-acetate and serum. Mol. Cell. Biol. 1987; 7:2201-2211.
27 Mijller R, Bravo R, Mijller D, Kurz C, Renz M. Different types of modification in c-fos and its associated protein p39: modulation of DNA binding by phosphoylation. Oncogene Res. 1987; 2:19-32.
28 Franza Jr. B R, Rauscher III F J, Josephs S F, Curran T. The Fos complex and Fos-related antigens recognize sequence elements that contain AP-1 binding sites. Science. 1988; 239:1150-1153.
29 Pulverer B J, Kyriakis J M, Avruch J, Nikolakaki E, Woodgett J R. Phosphorylation of c-jun mediated by MAP kinases. Nature. 1991; 353:670-674.
30 Franklin C C, Sanchez V, Wagner F, Woodgett J R, Kraft A S. Phorbol ester-induced amino-terminal phosphorylation of human JUN but not JUNB regulates transcriptional activation. Proc. Natl. Acad. Sci. USA. 1992; 89:7247-7251.
31 Minden A, Lin A, McMahon M, et al. Differential activation of ERK and JNK mitogen-activated protein kinases by Raf-1 and MEKK. Science. 1994; 266:1719-1723.
32 Cos O A, Chiariello M, Yu J-C, et al. The small GTP-binding proteins Rac1 and Cdc42 regulate the activity of the JNK/SAPK signaling pathway. Cell. 1995; 81:1137-1146.
33 Minden A, Lin A, Claret F-X, Abo A, Karin M. Selective activation of the JNK signaling cascade and c-Jun transcriptional activity by the small GTPases Rac and Cdc42Hs. Cell. 1995; 81:1147-1157.
34 Vandel L, Montreau N, Vial E, Pfarr C M, Binetruy B, Castellazzi M. Stepwise transformation of rat embryo fibroblasts:c-Jun, JunB, or JunD can cooperate with Ras for focus formation, but a c-Jun-containing heterodimer is required for immortalization. Mol. Cell. Biol. 1996; 16:1881-1888.
35 Harvey W H, Harb O S, Kosak S T, Sheaffer J C, Lowe L R, Heerema N A. Interferon-alpha-2b downregulation of oncogenes H-ras, c-raf-2, c-kit, c-myc, c-myb and c-fos in ESKOL, a hairy cell leukemic line, results in temporal perturbation of signal transduction cascade. Leukemia Res. 1994; 18:577-585.
36 Dallery E, Galiégue-Zouitina S, Collyn-d'Hooghe M, Quief S, Denis C, Hildebrand M P, Lantoine D, Deweindt C, Tilly H, Bastard C, Kerckaert J P. TTF, a gene encoding a novel small G protein, fuses to the lymphoma-associated LAZ3 gene by t(3,4) chromosomal translocation. Oncogene. 1995; 10:2171-2178.
37 Dallery-Prudhomme E, Roumier C, Denis C, Preudhomme C, Kerckaert J P, Galiégue-Zouitina S. Genomic structure and assignment of the RhoH/TTF small GTPase gene (ARHH) to 4p13 by in situ hybridization. Genomics. 1997; 1:89-94.
38 Li X, Bu X, Lu B, Avraham H, Flavell R A, Lim B. The hematopoietic-specific GTP-binding protein RhoH is GTPase deficient and modulates activities of other Rho GTPases by an inhibitory function. Mol. Cell. Biol. 2002; 22:1158-1171.
39 Gu Y, Jasti A C, Jansen M, Siefring J E. RhoH, a hematopoietic-specific Rho GTPase, regulates proliferation, survival, migration, and engraftment of hematopoietic progenitor cells. Blood. 2005; 105:1467-1475.
40 Kalyanaraman V S, Sarngadharan M G, Robert-Guroff M, Miyoshi I, Golde D, Gallo R C. A new subtype of human T-cell leukemia virus (HTLV-II) associated with a T-cell variant of hairy cell leukemia. Science. 1982; 218:571-573.
41 Foley G E, Lazarus H, Farber S, Uzman B G, Boone B A, McCarthy R E. Continuous culture of human lymphoblasts from peripheral blood of a child with acute leukemia. Cancer. 1965; 18:522-529.
42 Adams R A, Flowers A, Davis B J. Direct implantation and serial transplantation of human acute lymphoblastic leukemia in hamsters, SB-2. Cancer Res. 1968; 28:1121-1125.
43 Nadkarni J S, Nadkarni J J, Clifford P, Manolov G, Fenyo E M, Klein E. Characteristics of new cell lines derived from Burkitt lymphomas. Cancer. 1969; 23:64-79.
44. Pulvertaft J V. Cytology of Burkitt's tumor (African lymphoma). Lancet. 1964; 39:238-240.
44 Nilsson K, Bennich H, Johansson S G, Ponten J. Established immunoglobulin producing myeloma (IgE) and lymphoblastoid (IgG) cell lines from an IgE myeloma patient. Clin. Exp. Immunol. 1970; 7:477-489.
45 Kerckaert J P, Deweindt C, Tilly H, Quief S, Lecocq G, Bastard C. LAZ3, a novel zinc-finger encoding gene, is disrupted by recurring chromosome 3q27 translocations in human lymphomas. Nat. Genet. 1993; 5:66-70
46 Schiller J H, Bittner G, Meisner L F, Oberley T D, Norback D, Schwabe M, Faltynek C R, Raab-Traub N. Establishment and characterization of an Epstein-Barr virus spontaneously transformed lymphocytic cell line derived from a hairy cell leukemia patient. Leukemia. 1991; 5:399-407.
47 Faguet G B, Satya-Prakash K L, Agee J F. Cytochemical, cytogenetic, immunophenotypic and tumorigenic characterization of two hairy cell lines. Blood. 1988; 71:422-429.
48 Harvey W, Srour E F, Turner R, Carey R, Maze R, Starrett B, Kanagala R, Pereira D, Merchant P, Taylor M. Jansen J. Characterization of a new cell line (ESKOL) resembling hairy-cell leukemia: a model for oncogene regulation and late B-cell differentiation. Leuk. Res. 1991; 15:733-744.
49 Kontula K, Paavonen T, Vuopio P, Andersson L C. Glucocorticoid receptors in hairy-cell leukemia. Int. J. Cancer. 1982; 30:423-426.
51. Ades E W, Candal F J, Swerlick R A, George V G, Summers S, Bosse D C, Lawley T J. HMEC-1: establishment of an immortalized human microvascular endothelial cell line. J. Invest. Dermatol. 1992; 99:683-690.
50 Shelley C S, Farokhzad O C, Amaout M A. Identification of cell-specific and developmentally regulated nuclear factors that direct myeloid and lymphoid expression of the CD11a gene. Proc. Natl. Acad. Sci. USA. 1993; 90:5364-5368.
51 Shelley C S, Teodoridis J M, Park H, Farokhzad O C, Bottinger E P, Amaout M A. During Differentiation of the monocytic cell line U937, Puramediates induction of the CD11c gene promoter. J. Immunol. 2002; 168:3887-3893.
52 Gabert J, Beillard E, van der Velden V H, Bi W, Grimwade D, Pallisgaard N, Barbany G, Cazzaniga G, Cayuela J M, Cave H, Pane F, Aerts J L, De Micheli D, Thirion X, Pradel V, Gonzalez M, Viehmann S, Malec M, Saglio G, van Dongen J J. Standardization and quality control studies of “real-time” quantitative reverse transcriptase polymerase chain reaction of fusion gene transcripts for residual disease detection in leukemia—a Europe Against Cancer program. Leukemia. 2003; 17:2318-2357.
53 Lahousse S, Smorowski A L, Denis C, Lantoine D, Kerckaert J P, Galiégue-Zouitina S. Structural features of hematopoiesis-specific RhoH/ARHH gene: high diversity of 5′-UTR in different hematopoietic lineages suggests a complex post-transcriptional regulation. Gene. 2004; 343:55-68.
54 Chemy L K, Li X, Schwab P, Lim B, Klickstein L B. RhoH is required to maintain the integrin LFA-1 in a nonadhesive state on lymphocytes. Nat. Immunol. 2004; 5:961-967.
55 Kong T, Eltzschig H K, Karhausen J, Colgan S P, Shelley C S. Leukocyte adhesion during hypoxia is mediated by HIF-1 dependent induction of β2 integrin gene expression. Proc. Natl. Acad. Sci. USA. 2004; 101:10440-10445.
56 Corbi A L, López-Rodríguez C. CD11c integrin gene promoter activity during myeloid differentiation. Leuk and Lymph. 1997; 25:415-425.
57 Giehl K. Oncogenic Ras in tumour progression and metastasis. Biol. Chem. 2005; 386:193-205.
58 Akasaka T, Lossos I S, Levy R. BCL6 gene translocation in follicular lymphoma: a harbinger of eventual transformation to diffuse aggressive lymphoma. Blood. 2003; 102:1443-1448.
59 Preudhomme C, Roumier C, Hildebrand M P, Dallery-Prudhomme E, Lantoine D, Lai J L, Daudignon A, Adenis C, Bauters F, Fenaux P, Kerckaert J P, Galiégue-Zouitina S, Nonrandom 4p13 rearrangements of the RhoH/TTF gene, encoding a GTP-binding protein, in non-Hodgkin's lymphoma and multiple myeloma. Oncogene. 2000; 19:2023-2032.
60 Aamot H V, Micci F, Holte H, Delabie J, Heim S. G-banding and molecular cytogenetic analyses of marginal zone lymphoma. Br. J. Haematol. 2005; 130:890-901.
61 Liso A, Capello D, Marafioti T, Tiacci E, Cerri M, Distler V, Paulli M, Carbone A, Delsol G, Campo E, Pileri S, Pasqualucci L, Gaidano G, Falini B. Aberrant somatic hypermutation in tumor cells of nodular-lymphocyte-predominant and classic Hodgkin lymphoma. Blood. 2006; 108:1013-1020
62 Dijkman R, Tensen C P, Buettner M, Niedobitek G, Willemze R, Vermeer M H. Primary cutaneous follicular center lymphoma and primary cutaneous large B-cell lymphoma, leg type, are both targeted by aberrant somatic hypermutation but demonstrate differential expression of AID. Blood. 2006; 107; 4926-4929.
63 Pasqualucci L, Neumeister P, Goossens T, Nanjangud G, Chaganti R S, Kuppers R, Dalla-Favera R. Hypermutation of multiple proto-oncogenes in B-cell diffuse large-cell lymphomas. Nature. 2001; 412:341-346.
64 Reiniger L, Bodor C, Bognar A, Balogh Z, Csomor J, Szepesi A, Kopper L, Matolcsy A. Richter's and prolymphocytic transformation of chronic lymphocytic leukemia are associated with high mRNA expression of activation-induced cytidine deaminase and aberrant somatic hypermutation. Leukemia 2006; 20:1089-1095.
65 Montesinos-Rongen M, Van Roost D, Schaller C, Wiestler O D, Deckert M. Primary diffuse large B-cell lymphomas of the central nervous system are targeted by aberrant somatic hypermutation. Blood. 2004; 103:1869-1875.
66 Gaidano G, Pasqualucci L, Capello D, Berra E, Deambrogi C, Rossi D, Maria Larocca L, Gloghini A, Carbone A, Dalla-Favera R. Aberrant somatic hypermutation in multiple subtypes of AIDS-associated non-Hodgkin lymphoma. Blood. 2003; 102:1833-1841.
67 Southern E M. Detection of specific sequences among DNA fragments separated by gel electrophoresis. J. Mol. Biol. 1975; 98:503-517.

We claim:

1. A method of treating chronic lymphoid leukemia in a mammal comprising of determining the level of endogenous RhoH expression in the lymphocytes of said mammal and reconstituting RhoH gene expression in the chronic lymphoid leukemia lymphocytes if the endogenous RhoH expression is below a predetermined level.

2. The method of claim 1, wherein reconstituting RhoH gene expression comprises introducing an exogenous RhoH gene into said lymphocytes.

3. The method of claim 1, wherein the level of endogenous RhoH gene expression is determined by qRT-PCR.

4. The method of claim 3, wherein the exogenous Rho H gene is carried in an expression vector.

5. The method of claim 4, wherein the expression vector is a viral vector.

6. The method of claim 1, wherein the chronic lymphoid leukemia is selected from the group consisting of:
a. adult-T-cell leukemia (ATL);
b. chronic lymphocytic leukemia (CLL);
c. hairy cell leukemia (HCL).

7. The method of claim 1, wherein the mammal is a human.

8. The method of claim 1, wherein the level of endogeneous RhoH is reduced by at least 70% of the predetermined level.

9. A method of diagnosing chronic lymphoid leukemia in a mammal comprising of determining the level of RhoH expression in lymphocytes from the mammal, wherein a reduced expression of Rho H compared to a predetermined level indicates presence of chronic lymphoid leukemia.

10. The method of claim 9, wherein the level of endogenous RhoH gene expression is determined by qRT-PCR.

11. The method of claim 9, wherein the level of endogenous RhoH is reduced by at least 70% of the predetermined level.

12. A method of screening for compounds that alleviate RhoH repression in chronic lymphoid leukemia comprising of exposing ATL or HCL derived cell lines to the said compounds, determining the level of endogenous RhoH expression in said treated cell lines, and comparing the level with a predetermined level of endogenous RhoH expression, wherein a compound that results in an increase in the endogenous RhoH gene expression in the cell lines is considered a candidate compound for the treatment of chronic lymphoid leukemia.

13. The method of claim 12, wherein the cell lines carry a CD11c promoter-luciferase reporter vector or a RhoH promoter-luciferase reporter vector.

14. The method of claim 13, further comprising of determining the level of luciferase activity.

15. The method of claim 12, wherein the level of RhoH expression is determined by qRT-PCR.
